(19) United States
(12) Patent Application Publication
(10) Pub. No.: US 2016/0106676 A1
(43) Pub. Date: Apr. 21, 2016

(54) METHOD FOR THE PREPARATION OF DEGRADABLE MICROGEL PARTICLES, AND MICROGEL COMPOSITIONS THEREOF

(71) Applicant: McMaster University, Hamilton (CA)

(72) Inventors: Todd Hoare, Ancaster (CA); Daryl Sivakumaran, Dundas (CA); Madeline Simpson, Hamilton (CA); Eva Mueller, Oakville (CA)

(21) Appl. No.: 14/886,696

(22) Filed: Oct. 19, 2015

Related U.S. Application Data
(60) Provisional application No. 62/065,168, filed on Oct. 17, 2014.

(51) Int. Cl. A61K 9/16

(52) U.S. Cl. CPC A61K 9/1694; A61K 9/1635

(57) ABSTRACT

The present application relates to an aqueous method for the preparation of microgel particles using a stimuli-responsive prepolymer

### FIELD

The present application relates to an aqueous method for the preparation of microgel particles using a stimuli-responsive prepolymer.

### BACKGROUND

Microgels, solvent-swollen cross-linked gel particles with sub-micron diameters, have been widely investigated as potential materials of interest for wastewater treatment^{1,2}, catalysis³, and a myriad of biomedical and drug delivery applications⁴⁻⁷. Temperature-responsive microgels based on N-vinylcaprolactam (PVCL)⁸⁻¹⁰, N-isopropylacrylamide (NIPAM)^{11,12}, or poly(oligo(ethylene glycol)methacrylate) (POEGMA)¹³⁻¹⁵ have attracted particular attention as “smart”, switchable materials with the potential to dynamically change pore size, water content, and/or hydrophilicity upon heating above their volume phase transition temperature (VPTT). However, in the context of biomedical applications, the conventional precipitation based free-radical polymerization method used to make microgels with all the compositions listed above, offers challenges including metabolic clearance. The free radical polymerization strategy used results in non-degradable carbon-carbon backbones with molecular weights that are typically indeterminate. Given that renal clearance is typically possible only with molecular weight fragments no larger than 60,000 g/mol¹⁶, such materials are likely to accumulate within the liver and spleen, leading to potential chronic toxicity issues and a strong unlikelihood of any ultimate approval of such materials for clinical use.

One approach to address this challenge is to use a cross-linker which contains a degradable linkage between the cross-linking groups to prepare the microgel. The most popular degradable cross-linkers are based on disulfide linkages that can be cleaved in vivo by reduction^{9,14} (often facilitated by glutathione) and/or thiol exchange reactions. Diacrylate cross-linkers containing disulfide linkages have been used to prepare degradable microgels based on PVCL⁹ and POEGMA^{14,17}. Analogous strategies using polyvinylalkoxysiloxanes¹⁸, which degrade at basic conditions, or acetal groups, which can hydrolyze in acidic conditions¹⁹, have also been used to prepare thermoresponsive microgels that offer degradability. Alternately, degradability can be introduced by including degradable polymeric units directly into the microgel structure; for example, dextran-co-NIPAM microgels have been reported which can be degraded over time via oxidation and/or enzymatic degradation of the dextran component^{20,21}. However, if the goal is to ensure the ultimate clearance of the degradation products, the use of degradable cross-linkers is inherently limited as the molecular weight of the C—C backbone is not directly controllable using such an approach. While inclusion of chain transfer agents and/or controlled polymerization approaches (particularly reversible addition-fragmentation chain transfer polymerization or RAFT) can help to limit the molecular weight of the backbone, such approaches introduce additional complexity to the synthesis and, in the case of RAFT, may also limit the type of microgel functionalizations that are possible directly in the context of the polymerization process.

To counter these potential problems, microgels can instead be formed from well-defined polymeric precursors of controlled molecular weight and structure, with the microgel pre-polymers directly representing the degradation products. Several physically cross-linked microgels have been reported using this approach, primarily formed via self-assembly of block copolymers that contain one or more hydrophilic blocks (to facilitate stabilization of the nanoparticle following self-assembly) and thermoresponsive blocks (to drive self-assembly as the copolymer is heated). If a latent functionality is included in the thermoresponsive block, covalent cross-linking can occur following self-assembly to stabilize the microgel even following cooling; use of a cleavable functional group for this purpose can introduce degradability. Again, disulfide groups have been most widely reported in this context, with microgels based on poly(ethylene oxide)-block-(oligo(ethylene oxide) monomethyl ether methacrylate)²², polyethylene oxide-block-polystyrene²³, degradable polyester-block-POEGMA²⁴, and polysaccharide-grafted PNIPAM²⁵ formed using such an approach. Ionic interactions can also be used to assemble well-defined diblock copolymers in the same manner. For example, poly(ethylene oxide)-co-polyethyleneimine (PEO-PEI) polymers can be self-assembled into microgels via addition of anionic macromolecules²⁶. Alternately, solvent-based coacervation can be used to drive the formation of aggregates that can subsequently be cross-linked via bifunctional cross-linking agents²⁷, although this approach requires the use of solvents such as DMSO or DMF that can be difficult to fully remove and may induce downstream problems with in vivo use. Inverse emulsions can also be used to confine bulk gelation processes into a nanoparticulate form, although issues with organic solvent use, the generally poor monodispersity of the products, as well as the ultimate stability of the microgels produced can limit the utility of this approach.

Fabrication and Use of Functionalized Microgels

Microgels with environmental responses can be fabricated using a similar technique. From a biomedical applications perspective, microgels responsive to changes in pH and glucose concentration are of particular interest given the naturally-occurring gradients in both variables. For example, different locations within the body have varying pH gradients (e.g. stomach pH ˜1-1.5²⁸, lysosomes pH ˜4.5²⁹, mitochondrial matrix pH ˜7.5³⁰, and cancerous tissue pH 6.5-7³¹ relative to normal physiological pH 7.4³²). Similarly, the levels of glucose fluctuate depending on the timing of food-intake (before or after meals). pH variations in vivo can facilitate targeted responses of the microgel depending on microgel location within the body, while glucose responsive microgels can be used to monitor glucose levels in the blood stream for applications in triggered insulin delivery³³ or quantification of glucose concentrations within blood-protein mixtures³⁴.

pH-responsive microgels are typically prepared via copolymerization of monomers with a pKₐ in a targeted range for the desired smart response of the microgel vehicle. Ionization of these functional groups leads to swelling of the microgel due to Donnan equilibrium effects³⁵, facilitating pH-responsive control over microgel size and thus microgel pore size to allow for (as examples) entrapment and target release of molecules or drugs^{36,37} or a change in optics for the quantification of metabolites³⁸⁻⁴⁰. A number of pH microgels have been reported utilizing different polymer backbones and different comonomers. Hoare and Pelton developed a series of anionic PNIPAM-based microgels via copolymerization with different carboxylic acid containing comonomers⁴¹. Depending on the type of acidic monomer used (methacrylic acid, vinylacetic acid, or acrylic acid), changes in microgel size could be controlled over different pH ranges. Furthermore, the copolymerization kinetics of the comonomers relative to NIPAM were found to dictate the distribution of carboxylic acid groups within the microgel, further enabling tuning of the pH response; for example, vinylacetic acid was primarily incorporated at chain ends at or near the microgel surface via chain transfer processes and thus allowed for a larger and narrower range pH transition of the microgel than was observed for acrylic acid and methacrylic acid copolymer microgels^{42,43}. Basic monomers such as 2-(dimethylamino)ethyl methacrylate (DMAEMA), 4-vinylpyridine (VP), or N-3-dimethylaminopropyl methacrylate have also been copolymerized with NIPAM to provide a platform for pH sensitivity as well as cationic charge⁴⁴⁻⁴⁶. Copolymerizing multiple pH-ionizable comonomers with differing pKₐ ranges and/or copolymerization kinetics can further tune the microgel response achieved; for example, Bradley et al. used free radical precipitation polymerization to create NIPAM-based microgels with a core-shell configuration containing both DMAEMA and VP that exhibited a cationic charge below pH 6^{47,48}.

Drug delivery can be significantly aided by the presence of charge in the microgel network by promoting better uptake and subsequent control release of molecules from microgels. For example, cationic microgels have been shown to effectively bind and then release anionic surfactants^{47,48}. Acrylic acid functionalized PNIPAM microgels have been used to encapsulate the protein cytochrome C with greater efficiencies when the charge density of the microgel was high, promoting electrostatic binding and subsequent deswelling of the microgel⁴⁹. Similar observations were also made using anionically charged PNIPAM microgels for the uptake of cationic bupivacaine⁵⁰, with controlled release of bupivacaine achieved depending on the charge density of the microgel⁵¹. Electrostatic interactions between microgels of opposite charge can also be applied to create highly functional controlled release vehicles. For example, Gao et al.⁵² have developed drug trapping microgel aggregates via the formation of electrostatically-assembled hydrogels based on microgels copolymerized with acrylic acid (anionic) or N-[3-dimethylamino)propyl]methacrylamide (cationic). At neutral pH, the resulting hydrogels were able to trap a model drug (methylene blue) within the aggregate; upon changing the pH of the solution (to titrate the charge from one or the other microgel), the aggregate fell apart, releasing the drug into solution. Therefore, utilizing charged microgels allows for a number of different approaches to binding and releasing therapeutics in the field of drug delivery research.

Chemically-responsive microgels add an additional element of “smart” behaviour to microgels in that they can directly translate the presence or particular concentrations of specific metabolites (such as enzymes^{53,54}) in the body into a swelling response within the microgel. Glucose is a particularly interesting target molecule in this regard given that a glucose responsive microgel could possibly be used to quantify glucose concentrations and/or regulate in vivo delivery of insulin in response to blood glucose level changes³³. Such a technology is especially needed in the context of the rise in diabetic conditions among the aging population and would help minimize the possibility of patients experiencing an insulin overdose or hypoglycemia. Significant research effort has been invested into creating glucose sensitive microgels that utilize different glucose responsive functional groups. Wu et al.⁵⁵ created emulsion generated microgels which upon glucose oxidase exposure to glucose would result in a drop in localized pH; by using a pH degradable cross-linker, the microgel could then be degraded faster at higher glucose concentrations to enable triggered release of an insulin payload. Similarly, glucose-responsive microgels have been created utilizing the lectin concanavalin A, which upon glucose exposure results in the release of encapsulated insulin through a displacement mechanism in which glucose displaces the bound insulin conjugate⁵⁶. However, the major drawback with these approaches is that they are based on proteins and subsequently are limited by the potential toxicity, antigenicity, instability, and high cost of such ligands^{33,57}.

Alternatively, microgels based on glucose-responsive phenylboronic acid (PBA) groups can provide a cheaper and more stable option. PBA can form a reversible covalent bond with cis-diol groups in glucose (and other carbohydrates) to create a boronate ester^{33,58}, resulting in the generation of an anionic charge upon glucose complexation. As more glucose is added to the solution, more PBA-glucose complexation occurs and more boronate acid ionization occurs, leading to a shift in equilibrium of the trigonal boronic acid functional groups^{59,60}. This in turn allows more glucose to bind to PBA until equilibrium is reached at a certain glucose concentration. When incorporated into a microgel, this increase in charge associated with the PBA groups results in swelling of the microgel due to Donnan equilibrium effects⁵⁸, driving glucose-responsive microgel swelling. This strategy has been utilized by numerous research groups to create glucose responsive microgels based on NIPAM^{58,61,62}, sometimes copolymerized with other monomers such as 4-vinylpyridine⁶³ and acrylamide⁶⁴, that enable microgel responses to varying amounts of glucose. The microgels are also able to encapsulate a release payload (insulin most often) to provide a promising platform for glucose sensitive biomedical applications^{33,65}.

However, despite the promise of the smart responses engineered into the PNIPAM-based microgels described above, the conventional precipitation-induced free radical polymerization method widely used to prepare these microgels offers challenges in the context of the use of these microgels in biomedical applications. Since these free-radical polymerized microgels contain non-degradable carbon-carbon backbones, they are not clearable in vivo and are instead likely to accumulate within the liver or spleen (since renal clearance typically can only clear materials with molecular weight fragments no larger than 60,000 g/mol⁶⁶). The incorporation of degradable cross-linkers based on disulfides (cleavable via redox chemistry or thiol exchange reactions)^{67,68} or acetal groups (cleavable via hydrolysis)⁶⁹ into microgels could help with clearance of microgels from the body but still cannot effectively control the molecular weight between cross-links, potentially leaving degradation products that remain too large for effective renal clearance (like this current method can).

### SUMMARY

The present disclosure relates to an aqueous method for the preparation of microgel particles using a stimuli-responsive pre-polymer. Accordingly, the present application includes a method of preparing microgel particles, the method comprising,

(a) dissolving a stimuli-responsive pre-polymer in an aqueous solvent to form an aqueous solution;

(b) applying a stimulus to the aqueous solution to form a nanoaggregate pre-polymer; and

(c) adding a cross-linking polymer to the aqueous solution to crosslink the nanoaggregate pre-polymer to form the microgel particles,

wherein the stimuli-responsive pre-polymer is functionalized with cross-linkable moieties.

Further, the present application also includes a composition comprising microgel particles, wherein the microgel particles comprise,
(a) discrete nanoaggregate particles comprised of stimuli-responsive pre-polymers,
wherein the nanoaggregate particles are cross-linked with a cross-linking polymer;
wherein the microgel particles are biodegradable in vivo to reform the stimuli-responsive pre-polymers,
wherein the stimuli-responsive pre-polymers have a molecular weight lower than the renal clearance cut-off, and wherein the composition is substantially free of organic solvents.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1 shows images of self-assembled microgels following polymer cross-linking and cooling: (a) transmission electron microscopy image (100,000× magnification); (b) screenshot of nanoparticle tracking image demonstrating the monodispersity of microgels formed using this approach.

FIG. 2 shows the size distribution of nanoaggregates before cross-linking of a stimuli-responsive prepolymer and cross-linking polymer.

FIG. 3 shows particle size distributions of a microgel produced using a method of the disclosure (a) dynamic light scattering (intensity distribution, normalized to maximum intensity) and (b) nanoparticle tracking analysis (number distribution), demonstrating the high monodispersity of the microgel.

FIG. 4 shows the effect of temperature on a microgel preparation: (a) Lower critical solution temperature behavior of a stimuli-responsive prepolymer and cross-linking polymer; (b) Hydrodynamic diameter (as measured via dynamic light scattering) of microgels.

FIG. 5 shows the hydrodynamic diameter (measured by dynamic light scattering) of microgels produced using stir speeds between 200-650 RPM.

FIG. 6 shows the hydrodynamic diameter (from dynamic light scattering) of microgels produced using different mixing times.

FIG. 7 shows the hydrodynamic diameter (from dynamic light scattering) of self-assembled microgels prepared with varying concentrations of a stimuli-responsive prepolymer and cross-linking polymer.

FIG. 8 shows the particle size distributions (measured by dynamic light scattering) as a function of the reaction scale.

FIG. 9 shows the colloidal stability of a microgel prepared using the method of the disclosure: (a) Microgel size over the course of time (inset: Pictures of microgels immediately after preparation and following 5 months of storage at room temperature); (b) Pictures of microgels at equivalent concentrations (1 w/v %) before and after lyophilization.

FIG. 10 shows the degradation of a microgel prepared using the method of the disclosure: (a) Picture showing microgel suspension before and after acid treatment (1M HCl, 2 hours); (b) Gel permeation chromatography traces of degradation products from microgel hydrolysis (1M HCl, 24 hours exposure time).

FIG. 11 shows the hydrodynamic diameter versus temperature profiles (measured using dynamic light scattering) for microgels prepared with (a) different stimuli-responsive pre-polymer concentrations and a fixed mass-based ratio of cross-linking polymer; and (b) a fixed stimuli-responsive pre-polymer solution concentration and varying ratios of cross-linking polymer.

FIG. 12 shows the cell cytotoxicity to mouse 3T3 fibroblast cells (as measured via the MTT assay) relative to a cell-only (non-treated) control for precursor polymers and microgel.

FIG. 13 shows the particle size distributions measured by dynamic light scattering (intensity distribution, normalized to maximum intensity) of functionalized microgels prepared using a method of the disclosure.

FIG. 14 shows the LCST of functionalized stimuli-responsive pre-polymers.

FIG. 15 shows the aggregate size distributions measured by dynamic light scattering (intensity distribution, normalized to maximum intensity) of various stimuli-responsive pre-polymer.

FIG. 16 shows the changes in electrophoretic mobility of a functionalized microgel prepared using a method of the disclosure in response to changes in solution pH.

FIG. 17 shows the microgel hydrodynamic diameter change in response to changes in solution pH, for microgels prepared using a method of the disclosure and functionalized stimuli-responsive prepolymers.

FIG. 18 shows the potentiometric titration of a functionalized stimuli-responsive prepolymer relative to a saline blank.

FIG. 19 shows the microgel hydrodynamic diameter as a function of glucose concentration.

FIG. 20 shows the hydrodynamic diameter versus temperature profiles for microgels prepared using a method of the disclosure and functionalized stimuli-responsive prepolymer.

FIG. 21 shows the hydrodynamic diameter versus particle size for microgels produced via sequential addition of a stimuli-responsive prepolymer and cross-linking agent.

FIG. 22 shows the hydrodynamic diameter (a) and zeta potential (b) of amphoteric microgels prepared using a method of the disclosure.

FIG. 23 shows the ¹H NMR of cross-linking polymers.

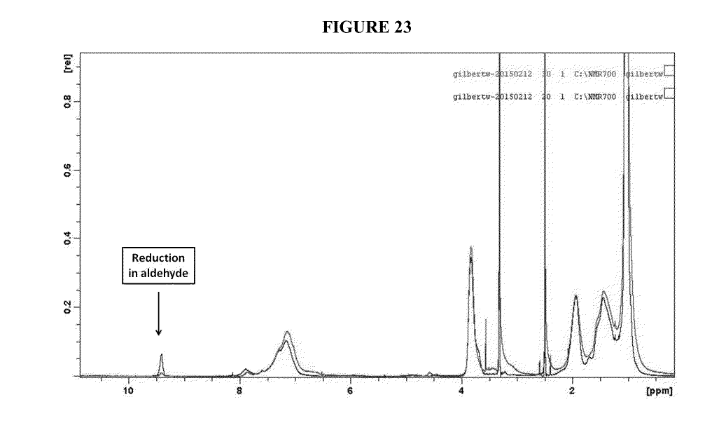

FIG. 24 shows, in one example of a stimuli-responsive pre-polymer, the confirmation of PBA grafting to PNIPAM-Hzd backbone.

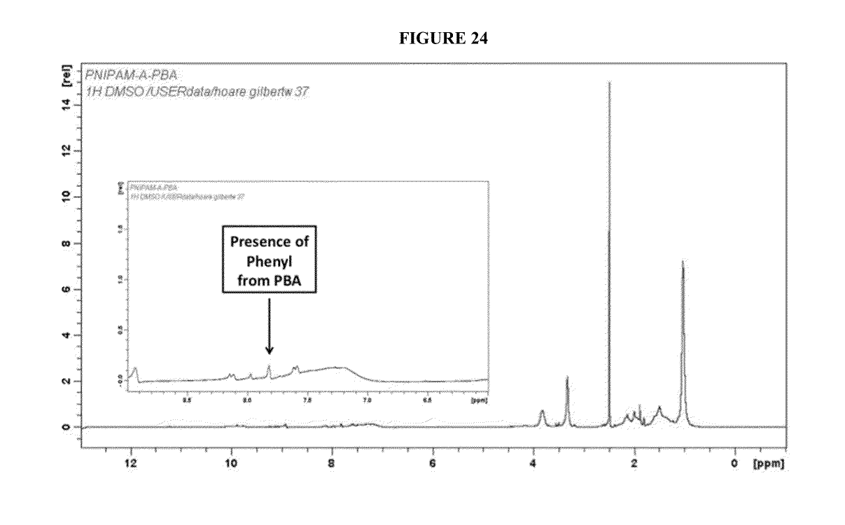

FIG. 25 illustrates, in one example of a stimuli-responsive prepolymer, the ¹H NMR confirmation of DMAEMA in PNIPAM-Hzd backbone.

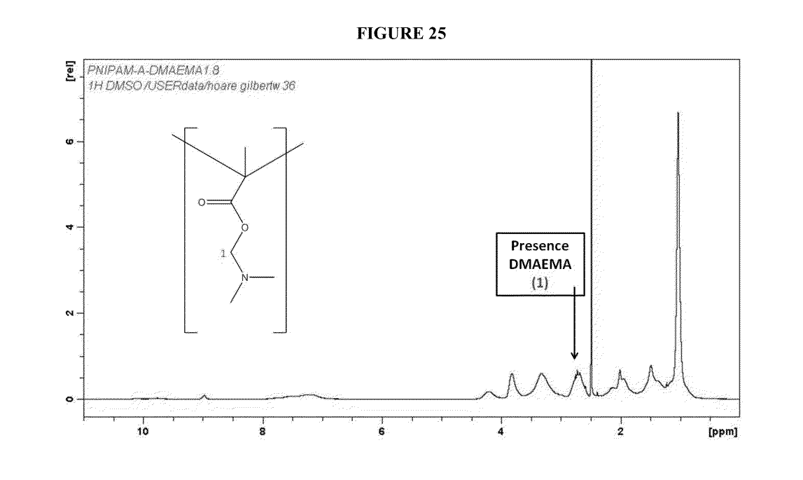

FIG. 26 shows the degree of ionization versus pH curve for a stimuli-responsive prepolymer (PNIPAM-Hzd-DMAEMA).

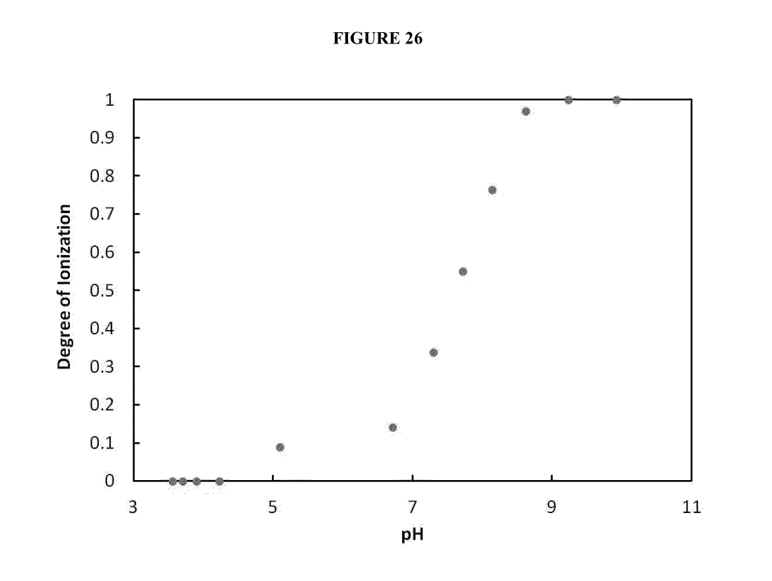

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The present description refers to a number of chemical terms and abbreviations used by those skilled in the art. Nevertheless, definitions of selected terms are provided for clarity and consistency.

As used in this application, the singular forms “a”, “an” and “the” include plural references unless the content clearly dictates otherwise. For example, an embodiment including “the polymer” should be understood to present certain aspects with one compound or two or more additional compounds.

In embodiments comprising an “additional” or “second” component, such as an additional or second polymer, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “suitable” as used herein means that the selection of the particular compound or conditions would depend on the specific synthetic manipulation to be performed, and the identity of the molecule(s) to be transformed, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The term “derivative” as used herein refers to a substance which comprises the same basic carbon skeleton and functionality as the parent compound, but can also bear one or more substituents or substitutions of the parent compound. For example, alkyl derivatives of acrylic acid would include any compounds in which an alkyl group is substituted on the acrylic acid backbone and would include methacrylic acid.

The term “monodisperse” as used herein describes a population of, for example, particles or polymers, where all of the constituents are the same or nearly the same size. For example, “monodisperse” refers to particle distributions in which 90%, or about 95%, or about 98%, or about 99%, or about 99.9%, or 100%, of the distribution lies within 15%, within 10% or within 5% of the mean particle size. Generally, a monodisperse particle population is considered to have a measured polydispersity value below 0.1 from Dynamic Light Scattering (DLS) and close to 1, measured through Nanoparticle Tracking Analysis (NTA).

The term “microgel particles” as used herein refers to particles of gel of any shape, formed of covalently cross-linked polymeric networks, having an average diameter of approximately 1 nm to 5 μm and which comprise cross-linked nanoaggregate pre-polymers, which are comprised of stimuli-responsive pre-polymers. The “microgel particles” dispersion of the present disclosure comprises microgel particles dispersed within an (aqueous) medium. Furthermore, the “microgel particles” of the present disclosure are able to swell or shrink in response to a variety of external stimuli such as temperature, pH, ionic strength, electric field and enzyme activities.

The terms “colloid” or “colloidal” as used herein refers to a suspension of finely divided particles in a continuous medium in which particles are approximately 5-5000 Å in size. Colloids are small enough that thermal energy drives their dynamics and ensures equilibration within a suspending fluid and they are also large enough that their position and motions can be measured precisely using optimal methods.

The terms “colloidal stability” or “colloidally stable” as used herein refers to colloidal particles which remain dispersed in liquids for long period of time (days to years).

The term “cross-linking polymer” as used herein refers to a polymer, which forms cross-links between or within the nanoaggregate pre-polymers due to a chemical reaction, in some cases, initiated by heat, pressure, change in pH, or radiation.

The terms “cross-linkable hydrophilic moieties” or “cross-linkable ionic moieties” as used herein refers to reactive chemical moieties that inherently comprise a charge (i.e., ion) or partial charge (i.e., hydrogen bonding character) such that they can be dissolved in polar solvents (i.e., water) and additionally can react with a complementary chemical moiety to form reversible covalent bonds.

The term “diameter” as used herein refers to either the physical diameter or hydrodynamic diameter. As used herein, the diameter may refer to the largest linear distance between two points on the surface of a particle. The particle diameter can be measured using a variety of techniques known in the art including, but not limited to, dynamic light scattering.

The term “stimuli-responsive pre-polymer” as used herein refers to polymers which can undergo a stimulus dependent conformational change. When cross-linked to form a microgel particle, this stimulus response has a consequential effect on the hydration of the particle, resulting in the formation of a nanoaggregate particle. By varying the stimulus, which includes, but is not limited to, changes in pH, temperature, ionic strength, and/or light, the microgel particles can transition between a collapsed configuration, in which the particle is in the compact configuration, or to a swollen configuration, in which the particle is in the form of a hydrated gel (or microgel).

The term “cross-linkable moieties” as used herein refers to moieties that are sufficiently reactive to undergo reactions with other complementary cross-linkable moieties.

The term “thermally responsive polymer” as used herein refers to polymers that undergo a temperature dependent conformational change which induces an effect on hydration of the particle.

The terms “lower critical solution temperature” or “LCST” as used herein refers to the critical temperature below which the stimuli-responsive pre-polymer becomes highly miscible with water. Above the LCST, the polymer is highly dehydrated and below the LCST, the pre-polymer is highly hydrated.

The term “aqueous solvent” as used herein refers to any solvent in which water comprises the majority of the solvent (typically from about 80%, to 99.9% water by weight), or pure water. The aqueous solvent optionally comprises, consists essentially of, or consists of water. While the aqueous solvent may comprise tap water, it preferably comprises processed water, such as deionized, distilled water or RO water (water subjected to reverse osmosis treatment), with no organic solvent or added solutes present. An exception may be the addition of small amounts of acidic, basic or buffer compounds for adjusting pH. Typically, the aqueous solvent is free, or substantially free, of organic solvents, such as methanol, ethanol, propanol, iso-propanol, tetrahydrofuran, etc., since these solvents are not desirable as residues in microgel particles that may be administered in vivo. However, if organic solvents are present, for example, ethanol, as a result of incomplete purification of the starting materials, they form part of the aqueous solvent in small amounts (less than 1%). A person skilled in the art would understand that tap water, can contain natural minerals, salts and/or other solutes, which would not affect the method of the disclosure.

The term “phase separates” as used herein refers to the conversion of a single-phase system into a multi-phasic system. For example, due to a chemical reaction, a dissolved substance may separate from its liquid medium (i.e. precipitate from the liquid medium). In the context of the present disclosure, the stimulus-responsive pre-polymer is at first in partial, or complete, dissolution in the aqueous medium and upon response to a stimulus, the formation of the nanoaggregate pre-polymers is a result of phase separation of the pre-polymers from the aqueous solution.

The term “w/w” as used herein means the number of grams of solute in 100 g of solution.

The term “w/v” as used herein refers to the number of grams of solution in 100 mL of solution.

The terms “nanoaggregate” or “nanoaggregate pre-polymer” as used herein is as an aggregate of a stimulus-responsive pre-polymer that has been subjected to a stimulus forming the nanoaggregate pre-polymer as a result of a phase separation, which have sizes that range, for example, from about 100 to about 800 nm.

### II. Method of the Application

The present disclosure is generally directed to a method for the preparation of degradable microgel particles, formed from a monodisperse, well-defined precursor polymer (the stimuli-responsive polymer) using an aqueous method that does not require any physical or chemical additive. As the microgel particles are formed from precursor polymers possessing certain molecular weights (for example, less than 50 kDa), the degradable microgel particles degrade back to their constituent precursor polymers having a size, for example, which is less than the renal clearance cut-off. In one embodiment, after, for example, drug delivery by the microgel administered in vivo, the degraded microgel particles (i.e. the stimuli-responsive prepolymer) can be cleared by the kidneys.

Accordingly, the present application is directed to a method of preparing microgel particles, the method comprising,

(a) dissolving a stimuli-responsive pre-polymer in an aqueous solvent to form an aqueous solution;

(b) applying a stimulus to the aqueous solution to form nanoaggregate pre-polymers; and

(c) adding a cross-linking polymer to the aqueous solution to crosslink the nanoaggregate pre-polymers to form the microgel particles

wherein the stimuli-responsive pre-polymer is functionalized with cross-linkable moieties.

In one embodiment, the aqueous solvent comprises water. In another embodiment, the aqueous solvent consists essentially of water, or consists of water.

In another embodiment, the stimuli-responsive pre-polymer phase separates in response to a stimulus selected from temperature, pH, ionic strength, or light conditions. In another embodiment, the stimuli-responsive pre-polymer is a thermally responsive polymer, a pH responsive polymer, an ionic strength responsive polymer or a light responsive polymer. In a further embodiment, the stimuli-responsive pre-polymer is a thermally responsive polymer.

In one embodiment, the stimuli-responsive pre-polymer is a thermally responsive polymer and the stimulus is an increase in temperature higher than the lower critical solution temperature (LCST). In one embodiment, the temperature is at the LCST, or about 5-10° C. higher, or about 5° C. higher than the LCST. In one embodiment, the temperature is between about room temperature, for example between about 20° C. and about 80° C. In one embodiment, the temperature (in part (b)) is increased to at least 50° Cm or at least about 70° C., or at least about 80° C.

In one embodiment, the stimuli-responsive pre-polymer is a copolymer, comprising:
a) a first monomer which is (N-isopropylacrylamide) (NIPAM), (oligo(ethylene glycol)methacrylate) (PEOGMA) and/or vinylcaprolactam; and
b) a second monomer which can be functionalized with cross-linkable moieties.

In another embodiment, the stimuli-responsive pre-polymer is a copolymer comprising a third monomer.

In one embodiment, the cross-linkable moieties form cross-linked bonds which are reversible.

In a further embodiment, the second monomer of the stimuli-responsive pre-polymer can be functionalized with cross-linkable hydrophilic or ionic moieties. In another embodiment, the second monomer of the stimuli-responsive pre-polymer is functionalized as a hydrazide moiety or an aldehyde moiety.

In another embodiment, the second monomer is acrylic acid, methacrylic acid, itaconic acid, fumaric acid, maleic acid, vinylacetic acid, or a derivative thereof or N-(2,2-dimethoxyethyl)methacrylamide (DMEMAm) or a derivative thereof.

In one embodiment, the second monomer is acrylic acid or a derivative thereof (AA), which forms a copolymer of, for example, NIPAM and AA, wherein at least a portion, or all, of the carboxylic acid groups of the acrylic acid are functionalized as hydrazide moieties. In another embodiment, when only a portion of the carboxylic acid groups of the acrylic acid are functionalized as hydrazide moieties, for example, the stimuli-responsive pre-polymer is anionic.

In one embodiment, the use of different monomers can impart different ionic character on the stimuli-responsive pre-polymer. For example, acrylic acid, methacrylic acid, itaconic acid, fumaric acid, maleic acid, vinylacetic acid or tert-butyl2-acryloylhydrazinecarboxylate (BAHC) can impart anionic character on the pre-polymer, while 2-dimethylaminoethylmethacrylate (DMAEMA) can impart cationic character on the pre-polymer.

In another embodiment, the second monomer is acrylic acid and the third monomer is DMAEMA, which forms a copolymer of, for example, NIPAM, AA and DMAEMA, wherein at least a portion, or all, of the carboxylic acid groups of the acrylic acid are functionalized as hydrazide moieties, while the DMAEMA imparts cationic character on the pre-polymer.

In another embodiment, the second monomer is acrylic acid and the third monomer is BAHC, which forms a copolymer of, for example, NIPAM, AA and BAHC, wherein at least a portion, or all, of the carboxylic acid groups of the acrylic acid are functionalized as hydrazide moieties, while the BAHC impart anionic character on the pre-polymer.

In another embodiment, the stimuli-responsive pre-polymer is comprised of a first monomer which is POEGMA and a second monomer which is DMEMAm or a derivative thereof, which forms a copolymer of, for example, POEGMA and DMEMAm, wherein at least a portion, or all, of the acetal groups of DMEMAm are functionalized with aldehyde moieties.

In another embodiment, the stimuli-responsive pre-polymer is further functionalized with groups which allow for the microgel particles to be responsive to chemical changes in the particular environment, such as glucose levels or pH levels. In one embodiment, for example, a stimuli-responsive pre-polymer comprised of a hydrazide functionalized copolymer of NIPAM and AA is further functionalized with a group which is responsive to changes in the concentration of glucose. For example, the copolymer is functionalized with 4-formylphenylboronic acid (PBA), which reacts with excess hydrazide groups to form a PBA functionalized stimuli-responsive pre-polymer. In another embodiment, the DMAEMA is a pH responsive groups.

In one embodiment, depending on the monomers, the stimuli-responsive pre-polymers can have anionic, cationic or amphoteric character.

In another embodiment, the stimuli-responsive pre-polymer has a molecular weight which is less than the molecular weight cut-off for renal (kidney) clearance. In another embodiment, the stimuli-responsive pre-polymer has a molecular weight which is less than about 60 kDa, or less than about 50 kDa. In another embodiment, the stimulus-responsive polymer has molecular weight of between about 10 to about 60 kDa, or about between about 20 to about 60 kDa. In one embodiment, the stimuli-responsive polymer forms nanoaggregates at temperatures above body temperature, and therefore, degradation of the microgel particles at body temperature thereby reforms the stimuli-responsive prepolymer (i.e. non-nanoaggregate form) which can be cleared by the kidney.

In one embodiment, the stimulus is a change in temperature, pH, ionic strength and/or light conditions. In one embodiment, the stimulus is a change in temperature. In another embodiment, the stimulus is in an increase in temperature above the lower critical solution temperature (LCST) of the stimulus-responsive polymer.

In one embodiment, about 0.15 w/v % to about 25.0 w/v %, about 0.15 w/v % to about 15.0 w/v %, about 0.15 w/v % to about 10.0 w/v %, about 0.15 w/v % to about 5.0 w/v %, or about 0.15 w/v % to about 2.5 w/v %, or about 0.2 w/v % to about 2 w/v %, of the stimuli-responsive pre-polymer is dissolved in the aqueous solvent. In another embodiment, the stimulus is applied to the aqueous solution for a period of about 1 to about 60 minutes, or about 2 to about 30 minutes, or about 2 to 10 minutes, or about 3 to about 8 minutes, or about 5 minutes to form the nanoaggregate particles.

In one embodiment, the cross-linking polymer is able to form cross-linking covalent bonds between, or within (intramolecularly or intermolecularly) the nanoaggregate pre-polymers. In another embodiment, the cross-linking polymer possesses reactive chemical functional groups which are complementary to the cross-linkable moieties on the stimuli-responsive pre-polymer and are able to cross-link the nanoaggregate pre-polymers.

In an embodiment, the cross-linking polymer is a second stimuli-responsive polymer. In another embodiment, the cross-linking polymer is a cross-linking stimuli-responsive polymer which is functionalized with moieties which are complementary to the cross-linkable moieties (for example, the hydrophilic or ionic) moieties of the stimuli-responsive pre-polymer.

In one embodiment, the cross-linking polymer is a copolymer, comprising:
a) a first monomer which is (N-isopropylacrylamide) (NIPAM), (oligo(ethylene glycol)methacrylate) and/or vinylcaprolactam; and
b) a second monomer which can be functionalized with cross-linkable moieties which are complementary to the cross-linkable moieties of the stimuli-responsive pre-polymer.

In another embodiment, the cross-linking polymer is a copolymer comprising a third monomer.

In another embodiment, the second monomer of the cross-linking polymer is functionalized with an aldehyde moiety or a hydrazide moiety (i.e. the cross-linkable moieties are an aldehyde moiety or a hydrazide moiety).

In another embodiment, the second monomer of the cross-linking polymer is N-(2,2-dimethoxyethyl)methacrylamide (DMEMAm) or a derivative thereof. In one embodiment, the second monomer is DMEMAm or a derivative thereof, which forms a copolymer of, for example, NIPAM and DMEMAm, wherein at least a portion, or all, of the acetal groups of DMEMAm are functionalized as aldehyde moieties.

In another embodiment, the second monomer is acrylic acid, methacrylic acid, itaconic acid, fumaric acid, maleic acid, or vinylacetic acid.

In another embodiment, the cross-linking polymer is comprised of a first monomer which is POEGMA or NIPAM and a second monomer which is acrylic acid or a derivative thereof, which forms a copolymer of, for example, POEGMA (or NIPAM) and AA, wherein at least a portion, or all, of the carboxylic groups of the AA are functionalized as hydrazide moieties.

In a further embodiment, the covalent cross-links are formed via reaction of hydrazide and aldehyde groups to form reversible hydrazone bonds. In an embodiment, the cross-linking polymer is responsive to the same or an alternate stimulus as the stimuli-responsive pre-polymer.

In one embodiment, the use of different monomers can impart different ionic character on the cross-linking polymer. For example, acrylic acid (or methacrylic acid, itaconic acid, fumaric acid, maleic acid, or vinylacetic acid) or BAHC can impart anionic character on the cross-linking polymer, while DMAEMA can impart cationic character on the cross-linking polymer. In another embodiment, the second monomer is DMEMAm and the third monomer is acrylic acid, which forms a copolymer of, for example, NIPAM, AA and DMEMAm, wherein at least a portion, or all, of the acetal groups of DMEMAm are functionalized as aldehyde moieties. In another embodiment, when the copolymers is comprised of NIPAM, DMEMAm, and DMAEMA and a portion, or all, of the acetal groups of DMEMAm are functionalized as aldehyde moieties, the copolymer has cationic character with the DMAEMA imparting cationic character.

In another embodiment, the cross-linking polymer is further functionalized with groups which allow for the microgel particles to be responsive to chemical changes in the particular environment, such as glucose levels or pH levels. In one embodiment, for example, a cross-linking polymer comprised of an aldehyde functionalized copolymer of NIPAM and DMEMAm is further functionalized with a group which is responsive to changes in the concentration of glucose. For example, the copolymer is functionalized with 3-aminophenylboronic acid (PBA), which proceeds through a reductive amination with excess aldehyde groups to form a PBA functionalized cross-linking polymer. In another embodiment, DMAEMA is used as a pH responsive monomer.

In one embodiment, depending on the monomers, the cross-linking polymers can have anionic, cationic or amphoteric character.

In another embodiment, the cross-linking polymer is added at a ratio of about 0.01 to about 0.3 (w/v), or about 0.05 to about 0.2 (w/v) as compared with the amount of stimuli-responsive pre-polymer added.

In one embodiment, the cross-linking bonds which bond the nanoaggregate pre-polymers together are degradable, such that after microgel particle formation, the cross-linking bonds are broken resulting in re-formation of the stimuli-responsive pre-polymers. In one embodiment, the cross-linking bonds are biodegradable or degradable in vivo. In one embodiment, the cross-linking bonds (i.e. the bonds stabilizing the nanoaggregate pre-polymers), fragment (or degrade) irreversibly, i.e., fragments cannot recombine in any manner or react with any other portion of the polymer to form a new covalent cross-link. In one embodiment, the cross-linking polymer forming the cross-linking bonds has the property of breaking down or degrading under certain conditions, e.g., at varying pH, temperature, ionic strength, electric stimuli or other environmental stimuli, or enzymatically.

In one embodiment, depending on the external environment, the microgel particle comprises less than about 70% (w/w) water, less than about 50% (w/w) of water, less than about 30% (w/w) of water or about 10% (w/w) of water. In this embodiment, such microgel particles are collapsed which refers to microgel particles which are substantially reduced in size and have a smaller average diameter than in the swollen state. In the collapsed state, the microgel particles adopt a configuration which does not favor the ingress of water into the particle. In another embodiment, depending on the environment, the microgel particles comprises about 70%(w/w) of water, about 85% (w/w) of water, about 90% w/w of water or about 95% (w/w) of water. In this embodiment, such microgel particles are swollen which refers to microgel particles that are substantially hydrated and enlarged, and have a greater average diameter than when the particle is in the collapsed state. It will be appreciated that this swelling is caused by a flow of water into the particle. It will be appreciated that the amount of water in the particle will depend on the temperature and/or pH as well the properties of the polymer comprising the microgel (e.g. charge density).

In an embodiment, due to the reversible nature of the cross-linking bonds, the microgel particles are degradable in vivo to reform the stimuli-responsive pre-polymers having the same, or similar, molecular weight prior to cross-linking. In one embodiment, the stimuli-responsive pre-polymers have a monodisperse molecular weight which is less than the molecular weight cut-off for renal (kidney) clearance. In another embodiment, the nanoaggregate pre-polymers have a monodisperse molecular weight which is less than about 60 kDa, or less than about 50 kDa. In another embodiment, the nanoaggregate particles have a monodisperse molecular weight of between about 10 to about 60 kDa, or between about 20 to about 60 kDa.

In another embodiment, the cationic microgel particles are comprised of hydrazide functionalized poly(N-isopropylacrylamide) and a copolymer of N-(2,2-dimethoxyethyl)methacrylamide appended from an aldehyde functionalized poly(N-isopropylacrylamide). In another embodiment, the anionic microgel particles are comprised of hydrazide functionalized poly(N-isopropylacrylamide) prepared as a copolymer with acrylic acid and phenylboronic acid. In a further embodiment, the amphoteric microgel particles are comprised of hydrazide functionalized poly(N-isopropylacrylamide) and a monomer selected from N-(2,2-dimethoxyethyl)methacrylamide, acrylic acid or phenylboronic acid appended from an aldehyde functionalized poly(N-isopropylacrylamide).

In an embodiment, the microgel particles have a particle size of about 70 nm to about 600 nm, or about 100 nm to about 500 nm, or about 200 to 400, or about 200 to about 300 nm, or about 220 to about 250 nm in diameter.

In an embodiment, the microgel particles are monodisperse, comprising a polydispersity of 0.05 to about 0.25 or about 0.1 to about 0.2. In another embodiment, the microgel particles comprise a polydispersity of 0.1. In another embodiment, the microgel particles can be lyophilized and stored without affecting the particle size or colloidal stability of the microgel.

In an embodiment, the microgel particles swell or deswell in response to pH and glucose concentrations depending on the monomers used for the stimuli-responsive pre-polymer and cross-linking polymer. In another embodiment, the microgel particles swell in response to pH of about 6 to about 10 for cationic microgel particles and a pH of about 4-7 for anionic microgel particles. In a further embodiment, the microgel particles swell in response to glucose concentrations from about 1 mg/mL to about 10 mg/mL.

In one embodiment, the microgels can also be lyophilized and redispersed without any change in colloidal stability or particle size and exhibit no significant cytotoxicity in vitro.

### III. Composition of the Application

The present application also includes a composition comprising microgel particles, wherein the microgel particles comprise,
(a) discrete nanoaggregate particles comprised of a stimuli-responsive pre-polymer, wherein the nanoaggregate particles are cross-linked with a cross-linking polymer;
wherein the microgel particles are biodegradable in vivo to reform the stimuli-responsive pre-polymer,
wherein the stimuli-responsive pre-polymers have a molecular weight lower than the renal clearance cut-off, and
wherein the composition is substantially free of organic solvents.

In one embodiment, the stimuli-responsive pre-polymer has a molecular weight which is less than the molecular weight cut-off for renal (kidney) clearance. In another embodiment, the stimuli-responsive pre-polymer has a molecular weight less than about, or less than about 60 kDa, or less than about 50 kDa. In another embodiment, the stimuli-responsive pre-polymer has a molecular weight of between about 10 to about 60 kDa, or about between about 20 to about 60 kDa.

In an embodiment, the stimulus-responsive polymer phase separates in response to a stimulus selected from temperature, pH, ionic strength, or light conditions. In another embodiment, the stimulus-responsive polymer is a thermally responsive polymer, a pH responsive polymer, an ionic strength responsive polymer or a light responsive polymer. In a further embodiment, the stimulus-responsive polymer is a thermally responsive polymer.

In one embodiment, the stimuli-responsive pre-polymer is a thermally responsive polymer and the stimulus is an increase in temperature higher than the lower critical solution temperature (LCST) of between about room temperature, for example between about 20° C. and about 80° C. In one embodiment, the temperature (in part (b)) is increased to at least 50° Cm or at least about 70° C., or at least about 80° C.

In one embodiment, the stimuli-responsive pre-polymer is a copolymer, comprising:
a) a first monomer which is (N-isopropylacrylamide) (NIPAM), (oligo(ethylene glycol)methacrylate) (PEOGMA) and/or vinylcaprolactam; and
b) a second monomer which can be functionalized with cross-linkable moieties.

In another embodiment, the stimuli-responsive pre-polymer is a copolymer comprising a third monomer.

In one embodiment, the cross-linkable moieties form cross-linked bonds which are reversible.

In a further embodiment, the second monomer of the stimuli-responsive pre-polymer can be functionalized with cross-linkable hydrophilic or ionic moieties. In another embodiment, the second monomer of the stimuli-responsive pre-polymer is functionalized as a hydrazide moiety or an aldehyde moiety.

In another embodiment, the second monomer is acrylic acid, methacrylic acid, itaconic acid, fumaric acid, maleic acid, vinylacetic acid, or a derivative thereof or N-(2,2-dimethoxyethyl)methacrylamide (DMEMAm) or a derivative thereof.

In one embodiment, the second monomer is acrylic acid or a derivative thereof (AA), which forms a copolymer of, for example, NIPAM and AA, wherein at least a portion, or all, of the carboxylic acid groups of the acrylic acid are functionalized as hydrazide moieties. In another embodiment, when only a portion of the carboxylic acid groups of the acrylic acid are functionalized as hydrazide moieties, for example, the stimuli-responsive pre-polymer is anionic.

In one embodiment, the use of different monomers can impart different ionic character on the stimuli-responsive pre-polymer. For example, acrylic acid, methacrylic acid, itaconic acid, fumaric acid, maleic acid, vinylacetic acid or tert-butyl2-acryloylhydrazinecarboxylate (BAHC) can impart anionic character on the pre-polymer, while 2-dimethylaminoethylmethacrylate (DMAEMA) can impart cationic character on the pre-polymer.

In another embodiment, the second monomer is acrylic acid and the third monomer is DMAEMA, which forms a copolymer of, for example, NIPAM, AA and DMAEMA, wherein at least a portion, or all, of the carboxylic acid groups of the acrylic acid are functionalized as hydrazide moieties, while the DMAEMA imparts cationic character on the pre-polymer.

In another embodiment, the second monomer is acrylic acid and the third monomer is BAHC, which forms a copolymer of, for example, NIPAM, AA and BAHC, wherein at least a portion, or all, of the carboxylic acid groups of the acrylic acid are functionalized as hydrazide moieties, while the BAHC impart anionic character on the pre-polymer.

In another embodiment, the stimuli-responsive pre-polymer is comprised of a first monomer which is POEGMA and a second monomer which is DMEMAm or a derivative thereof, which forms a copolymer of, for example, POEGMA and DMEMAm, wherein at least a portion, or all, of the acetal groups of DMEMAm are functionalized with aldehyde moieties.

In another embodiment, the stimuli-responsive pre-polymer is further functionalized with groups which allow for the microgel particles to be responsive to chemical changes in the particular environment, such as glucose levels or pH levels. In one embodiment, for example, a stimuli-responsive pre-polymer comprised of a hydrazide functionalized copolymer of NIPAM and AA is further functionalized with a group which is responsive to changes in the concentration of glucose. For example, the copolymer is functionalized with 4-formylphenylboronic acid (PBA), which reacts with excess hydrazide groups to form a PBA functionalized stimuli-responsive pre-polymer. In another embodiment, the DMAEMA is a pH responsive groups.

In one embodiment, depending on the monomers, the stimuli-responsive pre-polymers can have anionic, cationic or amphoteric character.

In one embodiment, the cross-linking polymer is able to form cross-linking covalent bonds between, or within (intramolecularly or intermolecularly) the nanoaggregate particles. In another embodiment, the cross-linking polymer possesses reactive chemical functional groups which are complementary to the cross-linkable moieties on the stimuli-responsive pre-polymer and are able to cross-link the nanoaggregate particles.

In an embodiment, the cross-linking polymer is a second stimuli-responsive polymer. In another embodiment, the cross-linking polymer is a cross-linking stimuli-responsive polymer which is functionalized with moieties which are complementary to the cross-linkable moieties (for example, the hydrophilic or ionic) moieties of the stimuli-responsive pre-polymer.

In one embodiment, the cross-linking polymer is a copolymer, comprising:
a) a first monomer which is (N-isopropylacrylamide) (NIPAM), (oligo(ethylene glycol)methacrylate) and/or vinylcaprolactam; and
b) a second monomer which can be functionalized with cross-linkable moieties which are complementary to the cross-linkable moieties of the stimuli-responsive pre-polymer.

In another embodiment, the cross-linking polymer is a copolymer comprising a third monomer.

In another embodiment, the second monomer of the cross-linking polymer is functionalized with an aldehyde moiety or a hydrazide moiety (i.e. the cross-linkable moieties are an aldehyde moiety or a hydrazide moiety).

In another embodiment, the second monomer of the cross-linking polymer is N-(2,2-dimethoxyethyl)methacrylamide (DMEMAm) or a derivative thereof. In one embodiment, the second monomer is DMEMAm or a derivative thereof, which forms a copolymer of, for example, NIPAM and DMEMAm, wherein at least a portion, or all, of the acetal groups of DMEMAm are functionalized as aldehyde moieties.

In another embodiment, the second monomer is acrylic acid, methacrylic acid, itaconic acid, fumaric acid, maleic acid, or vinylacetic acid.

In another embodiment, the cross-linking polymer is comprised of a first monomer which is POEGMA or NIPAM and a second monomer which is acrylic acid or a derivative thereof, which forms a copolymer of, for example, POEGMA (or NIPAM) and AA, wherein at least a portion, or all, of the carboxylic groups of the AA are functionalized as hydrazide moieties.

In a further embodiment, the covalent cross-links are formed via reaction of hydrazide and aldehyde groups to form reversible hydrazone bonds. In an embodiment, the cross-linking polymer is responsive to the same or an alternate stimulus as the stimuli-responsive pre-polymer.

In one embodiment, the use of different monomers can impart different ionic character on the cross-linking polymer. For example, acrylic acid (or methacrylic acid, itaconic acid, fumaric acid, maleic acid, or vinylacetic acid) or BAHC can impart anionic character on the cross-linking polymer, while DMAEMA can impart cationic character on the cross-linking polymer. In another embodiment, the second monomer is DMEMAm and the third monomer is acrylic acid, which forms a copolymer of, for example, NIPAM, AA and DMEMAm, wherein at least a portion, or all, of the acetal groups of DMEMAm are functionalized as aldehyde moieties. In another embodiment, when the copolymers is comprised of NIPAM, DMEMAm, and DMAEMA and a portion, or all, of the acetal groups of DMEMAm are functionalized as aldehyde moieties, the copolymer has cationic character with the DMAEMA imparting cationic character.

In another embodiment, the cross-linking polymer is further functionalized with groups which allow for the microgel particles to be responsive to chemical changes in the particular environment, such as glucose levels or pH levels. In one embodiment, for example, a cross-linking polymer comprised of an aldehyde functionalized copolymer of NIPAM and DMEMAm is further functionalized with a group which is responsive to changes in the concentration of glucose. For example, the copolymer is functionalized with 3-aminophenylboronic acid (PBA), which proceeds through a reductive amination with excess aldehyde groups to form a PBA functionalized cross-linking polymer. In another embodiment, DMAEMA is used as a pH responsive monomer.

In one embodiment, depending on the monomers, the cross-linking polymers can have anionic, cationic or amphoteric character.

In one embodiment, the cross-linking bonds are reversible.

In one embodiment, the cross-linking bonds which bond the nanoaggregate particles together are degradable, resulting in release of the stimuli-responsive pre-polymers. In one embodiment, the cross-linking bonds are biodegradable or degradable in vivo. In one embodiment, the cross-linking bonds (i.e. the bonds stabilizing the nanoaggregates, fragment (or degrade) irreversibly, i.e., fragments cannot recombine in any manner or react with any other portion of the polymer to form a new covalent cross-link. In one embodiment, the cross-linking agent forming the cross-linking bonds has the property of breaking down or degrading under certain conditions, e.g., at varying pH, temperature, ionic strength, electric stimuli or other environmental stimuli, or enzymatically.

In one embodiment, the microgel particles comprise between about 20% (w/w) water and 99% (w/w) water.

In an embodiment, due to the reversible or degradable nature of the cross-linking bonds, the microgel particles are degradable in vivo to reform the nanoaggregate particles (i.e. stimuli-responsive pre-polymer) having the same, or similar, molecular weight compared to the non-crosslinked particles. In one embodiment, the reformed nanoaggregate particles have a molecular weight which is less than the molecular weight cut-off for renal (kidney) clearance. In another embodiment, the nanoaggregate particles have a molecular weight which is less than about 60 kDa, or less than about 50 kDa. In another embodiment, the reformed nanoaggregate particles have a molecular weight of between about 10 to about 60 kDa, or about between about 20 to about 60 kDa.

In an embodiment, the microgel particles comprise cationic, anionic or amphoteric character. In another embodiment, the cationic microgel particles are comprised of hydrazide functionalized poly(N-isopropylacrylamide) and N-(2,2-dimethoxyethyl)methacrylamide appended from an aldehyde functionalized poly(N-isopropylacrylamide). In another embodiment, the anionic microgel particles are comprised of hydrazide functionalized poly(N-isopropylacrylamide) and a monomer selected from acrylic acid and phenylboronic acid appended from an aldehyde functionalized poly(N-isopropylacrylamide). In a further embodiment, the amphoteric microgel particles are comprised of hydrazide functionalized poly(N-isopropylacrylamide) and a monomer selected from N-(2,2-dimethoxyethyl)methacrylamide, acrylic acid or phenylboronic acid appended from an aldehyde functionalized poly(N-isopropylacrylamide).

In an embodiment, the microgel particles have a particle size of about 70 nm to about 600 nm, or about 100 nm to about 500 nm, or about 200 to 400, or about 200 to about 300 nm, or about 220 to about 250 nm in diameter.

In an embodiment, the microgel particles are monodisperse, comprising a polydispersity of 0.05 to about 0.25 or about 0.1 to about 0.2. In another embodiment, the microgel particles comprise a polydispersity of about 0.1. In another embodiment, the microgel particles can be lyophilized and stored without affecting the particle size or colloidal stability of the microgel.

In an embodiment, the microgel particles swell or deswell in response to pH and glucose concentrations depending on the monomers used for the stimuli-responsive pre-polymer and cross-linking polymer. In another embodiment, the microgel particles swell in response to pH of about 6 to about 10 for cationic microgel particles and a pH of about 4-7 for anionic microgel particles. In a further embodiment, the microgel particles swell in response to glucose concentrations from about 1 mg/mL to about 10 mg/mL.

In one embodiment, the composition is substantially free, or free, of organic solvents. In an embodiment, the composition is about 98%, about 99%, about 99.9% or about 100% free of organic solvents. In another embodiment, the composition is about 99% free of organic solvents. In a further embodiment, the composition is about 99.9% free of organic solvents.

In one embodiment of the disclosure, the microgel particles of the present disclosure are useful biomedical applications such as drug delivery vehicles, biosensors, molecular probes, mechanical supports for soft tissue, biological lubricants, and other applications. In another embodiment, the degradability (for example, acidic degradability) of the microgel particles result in microgels which are useful as intracellular drug delivery vehicles (i.e. degradation would happen inside the endosome but not outside the cell).

In another embodiment, the microgels of the present disclosure are useful in environmental applications, such as avoiding potential concerns with long-term ecosystem nanoparticle toxicity in applications such as enhanced oil recovery, water remediation, environmental biosensing, bioseparation applications, as the microgel (as a support substrate) can be degraded away to expose the bound analyte(s). In another embodiment, the microgels are useful as tunable optical devices, or dissolvable mechanical switches in nanofluidic applications.

### EXAMPLES

The following non-limiting examples are illustrative of the present application:

### Example 1

### Synthesis of Starting Polymers and Preparation of Microgels

Synthesis of N-(2,2-dimethoxyethyl)methacrylamide (DMEMAm) Monomer:

The acetal-containing comonomer N-(2,2-dimethoxyethyl)methacrylamide (DMEMAm) was synthesized using protocols developed by Smeets et al⁷¹. Briefly, 50 mL of aminoacetaldehyde dimethylacetal and 100 mg of TEMPO were dissolved in 100 mL of a 5M sodium hydroxide solution at 10° C. A total of 47 mL of methacryloyl chloride was then added drop-wise over a period of 2 hours, and the resulting mixture was left to react for 24 hours at room temperature under a nitrogen purge. This mixture was then extracted with 150 mL of petroleum ether to remove impurities. The aqueous phase was saturated with sodium chloride and extracted three times with 75 mL aliquots of tert-butyl methyl ether. The organic phase was dried with magnesium sulfate, filtered, and concentrated under reduced pressure.

Synthesis of Hydrazide Functionalized PNIPAM (PNIPAM-Hzd):

Hydrazide-functionalized PNIPAM was produced through single site attachment of a large (five-fold) molar excess of adipic acid dihydrazide to acrylic acid functionalized PNIPAM via carbodiimide coupling chemistry. Briefly, acrylic acid functionalized PNIPAM was prepared by polymerizing 4.5 g NIPAM and 0.5 g acrylic acid (15 mol % total monomer) using 0.056 g 2,2-azobisisobutyric acid dimethyl ester (AIBME) as the initiator and 80 μL thioglycolic acid as the chain transfer agent in 20 mL of absolute ethanol overnight at 56° C. under nitrogen. The solvent was then removed by evaporation, and the polymer was dissolved in 100 mL of Milli-Q water and dialysed against Milli-Q water over six, six-hour cycles. The PNIPAM-co-AA oligomer was then modified with hydrazide groups by dissolving 1 g of PNIPAM-co-AA, 20 g of adipic acid dihydrazide, and 11 g of EDC in 200 mL of Milli-Q water. The pH was adjusted to 4.75 and maintained throughout the reaction via addition of 0.1 M HCl as required to facilitate conjugation of ADH to acrylic acid groups. The resulting solution was dialysed against Milli-Q water over six, six-hour cycles (12-14 kDa MWCO) and lyophilized. Conductometric titration (ManTech Inc.) indicated that 95 mol % of acrylic acid monomer units in the polymer were functionalized with a hydrazide group using this approach.

Synthesis of Aldehyde Functionalized PNIPAM (PNIPAM-Ald):

Aldehyde-functionalized PNIPAM was prepared via copolymerization of NIPAM and DMEMAm followed by hydrolysis of the acetal groups in DMEMAm. Briefly, 4 g of NIPAM, 0.95 g of DMEMAm (13.4 mol % total monomer), 0.056 g AIBME initiator, and 80 μL of thioglycolic acid chain transfer agent were dissolved in 20 mL of absolute ethanol and heated to 56° C. under a nitrogen purge. After polymerization, solvent was removed by evaporation and the resulting polymer redissolved in 1M HCl and allowed to react for 24 hours to convert the pendant acetal groups in DMEMAm into aldehyde groups. The resulting polymer was then purified via six, six-hour dialysis cycles (12-14 kDa MWCO) and lyophilized for storage. Silver ion titration⁷² indicated that 12 mol % of monomers in the polymer were functionalized with an aldehyde group.

Polymer Lower Critical Solution Temperature Measurements:

The lower critical solution temperature (LCST) of the precursor polymers was measured using UV-vis spectrophotometry by identifying the temperature at which the transmittance of the sample was 95%.

Microgel Self-Assembly:

For the base recipe, microgels were made by first preparing stock solutions of PNIPAM-Hzd (1 w/v %) and PNIPAM-Ald (1 w/v %) in separate deionized water solutions. A volume of 5 mL of the PNIPAM-Hzd stock solution was heated to 70° C. for ˜5 minutes until the polymer solution became opaque (i.e. above the lower critical solution temperature of PNIPAM-Hzd) under magnetic stirring (350 RPM). A 0.25 mL aliquot of PNIPAM-Ald (5-20 wt % of the mass of PNIPAM-Hzd present in the seed solution) was then added drop-wise into the solution over a period of 5-10 seconds and allowed to stir for an 15 minutes. Microgel solutions were then removed from the temperature-controlled oil bath and allowed to cool at room temperature overnight. Additional experiments were performed by adjusting (one at a time) the temperature (50° C., 60° C., 70° C.), the stir rate (200-650 RPM), the time of mixing prior to cooling (1-60 minutes), the concentration of PNIPAM-Hzd in the seed solution (0.2 w/v %, 0.5 w/v % and 2 w/v %) and the mass ratio of PNIPAM-Hzd:PNIPAM-Ald (prepared by increasing the concentration of the PNIPAM-Ald stock solution to 2 w/v % (10 wt % of PNIPAM-Hzd), 3 w/v % (15 wt % of PNIPAM-Hzd), and 4 w/v % (20 wt % of PNIPAM-Hzd)) while maintaining the volumes of each solution added. The Ald:Hzd ratios given in the results represent the mass ratio of aldehyde:hydrazide polymers used to prepare the microgel using this addition procedure.

Larger batch sizes of up to 50 mL were also produced by keeping the stock solution concentrations and the relative volumes of the solutions added the same but scaling the total volumes used by the factors indicated (2×, 4×, and 10×). Acrolein (100 μL, 450× molar excess) was added to select solutions to quench unreacted hydrazide groups. Selected samples were also dialyzed over six, six-hour cycles (3500 kDa MWCO), lyophilized, and then resuspended at the same 1 w/v % concentration in Millipore water to assess microgel redispersibility.

Transmission Electron Microscopy:

Morphology characterization of microgels was performed using transmission electron microscopy (TEM, JEOL 1200EX TEMSCAN). A 3.5 ul aliquot of microgels was deposited onto a Cu/Rh coated Maxtaform™ grid. An accelerating voltage of 100 kV was used.

Microgel Particle Size Measurement:

Microgel particle sizes were assessed using two different techniques. Dynamic light scattering measurements were performed using a Brookhaven 90Plus Particle Analyser running Particle Solutions Software (Version 2.6, Brookhaven Instruments Corporation). Detection of scattering was completed using a 659 nm laser at a 90° angle configuration. Microgel concentrations were adjusted until an intensity of approximately between 200 to 500 kilocounts per second was achieved. Each measurement was conducted over 2 minutes and was repeated at least 4 times, with intensity-weighted particle sizes and particle size distributions reported as averages plus or minus standard deviations of these replicates. The temperature dependence of microgel size was assessed using the same method by ramping the temperature at 2° C. intervals over the temperature range of 25° C. to 70° C., using a stabilization period of 5 minutes for equilibration at each temperature. Alternately, single nanoparticle tracking analysis (NTA) assessments of particle size were conducted using a NanoSight instrument (NanoSight NTA 2.3). Samples were diluted 400-fold from the microgel stock solution in MilliQ water prior to measurement. Measurements were conducted at 25° C. for duration of 60 seconds, with number-weighted microgel sizes and distributions reported.

Electrophoretic Mobility:

Electrophoretic mobility measurements were conducted using a ZetaPlus zeta potential analyzer (Brookhaven Instruments Corporation) operating in PALS (phase analysis light scattering) mode (V.2.5). Samples were prepared at concentrations of ˜2.5 mg/mL in 10 mM KCl, with 5 replicates consisting of 30 cycles per replicate (150 total runs) used per sample tested; the experimental uncertainties represent the standard error of the replicate measurements.

Microgel Degradation:

Microgel solutions were exposed to hydrochloric acid solutions to assess the potential degradability of the microgels as well as characterize the ultimate degradation products. A volume of 5 mL of 1 M hydrochloric acid was added to 1 mL of a 10 mg/mL microgel suspension. Size measurements were made at various times using single nanoparticle tracking analysis to determine changes in particle size and particle count as a function of time, reflective of microgel degradation. Gel permeation chromatography using a Waters 590 HPLC pump, three Waters Styragel columns (HR2, HR3, HR4; 30 cm×7.8 mm (i.d.); 5 μm particles) at 40° C., a Waters 410 refractive index detector operating at 35° C., and DMF as the solvent was used to measure the molecular weight of the precursor polymers as well as the degradation products following 24 hours of treatment with 1M HCl to ensure break-down of the microgels into its precursor polymer components.

Microgel Cell Viability:

The viability of cells in response to precursor polymer and microgel exposure was completed using 3T3 Mus musculus fibroblasts. Cell viability was assayed in proliferation media consisting of 500 mL DMEM, 50 mL FBS, and 5 mL PS. Test were conducted in polystyrene 24 well plates (10,000 cells/well) by exposing cells to microgel concentrations ranging from 0.1 mg/mL to 2 mg/mL for a period of 24 hours. Viability was assessed using the metabolically based thiazolyl blue tetrazolium bromide (MTT) assay, modified from manufacturer's protocols as described by Pawlikowska⁷³. The concentration of the solubilized formazan product was measured at 540 nm using a microplate reader (Biorad, Model 550) relative to a control well containing the cells but not exposed to the microgels. Each sample was tested 4 times each to ensure consistent behavior among the cell response, with the error bars representing the standard deviation of the measurements.

Results & Discussion

(i) Microgel Fabrication

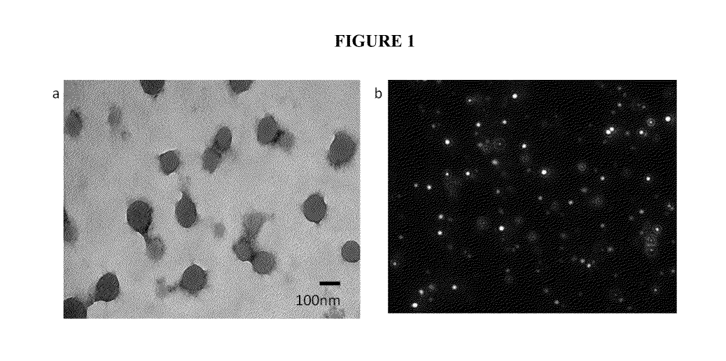

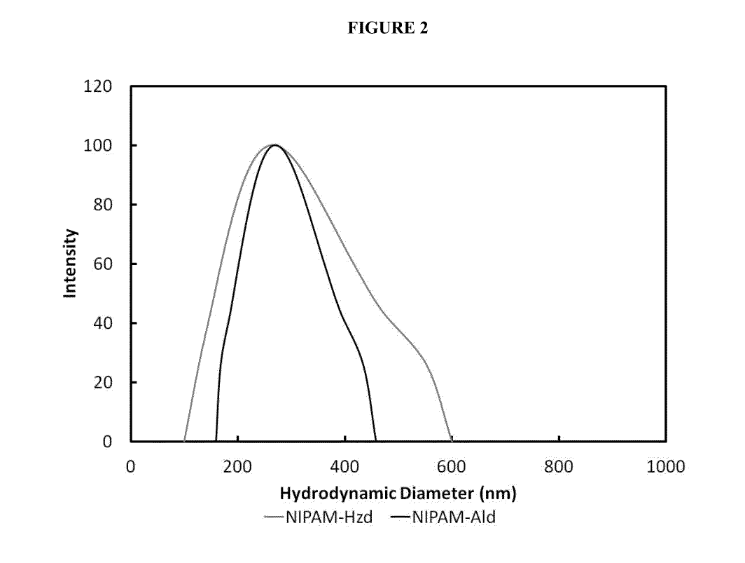

Microgels were formed using polymeric precursors functionalized with hydrazide and aldehyde functional groups as the building blocks and the thermal phase transition in water as the mechanism to drive nanoparticle formation. The PNIPAM-Hzd solution was initially heated up above its lower critical solution temperature (LCST) to form a thermally-collapsed nanoaggregate whereas the PNIPAM-Ald polymer was used as a cross-linker to stabilize the aggregates post-formation to create stable microgel particles. Support for this mechanism of particle formation was obtained by dynamic light scattering data on the PNIPAM-Hzd nanoaggregates prior to PNIPAM-Ald addition, which indicates that the size of the PNIPAM-Hzd nanoaggregates (266+1.5 nm at 1 mg/mL concentration, FIG. 2) directly templates the size of the resulting microgels at the same temperature (FIG. 1 and Table 1). This is in contrast to the conventional precipitation-based mechanism of forming conventional PNIPAM microgels in which pre-polymers grow in solution, undergo a phase transition, and then sequentially precipitate on to a seed particle to form a microgel⁷⁴. Cooling of the nanoaggregate suspension without adding cross-linker results in complete re-dissolution of the polymer (i.e. no signal is observed using dynamic light scattering); in contrast, after the aldehyde-PNIPAM was added, colloidally stable nanoparticles were maintained.

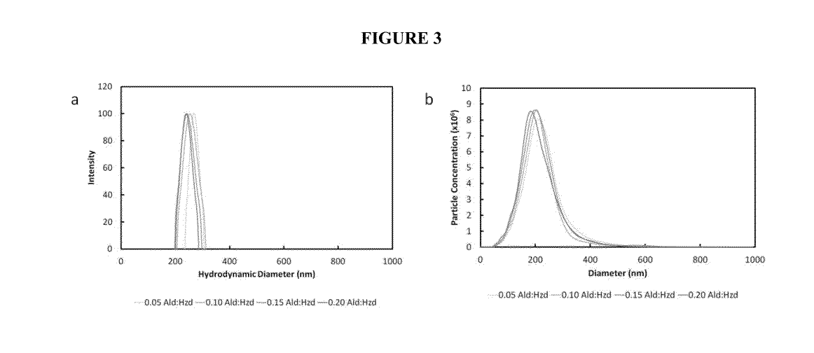

For the base microgel (1 wt % PNIPAM-Hzd, Ald:Hzd=0.10, 70° C. fabrication temperature, 350 RPM mixing, 15 minute mixing time), TEM images indicate that the microgels were consistently spherical (FIG. 1a), and nanoparticle tracking analysis conducted at room temperature confirmed the uniformity of the resulting microgel suspension (FIG. 1b). The polydispersity of the microgels produced from this process is low. FIG. 3 shows the full particle size distributions of microgels produced using various Ald:Hzd polymer ratios as measured using dynamic light scattering (intensity-based distribution, FIG. 3a) and nanoparticle tracking analysis (single particle number-based distribution, FIG. 3b), while Table 1 shows the mean particle sizes and polydispersity values measured using each technique.

The polydispersity of the microgels is narrow, independent of the polymer concentration used to prepare the microgels, with a highly reproducible, single size intensity peak appearing in both DLS measurements and nanoparticle tracking measurements. Measured polydispersity values from DLS results were in all cases below 0.1, generally considered to indicate a monodisperse particle population⁷⁵. In the case of polydispersity calculated from NTA measurements, the value of D_{w}/Dₙ is close to 1, indicating the microgels sized are largely monodisperse. Given the importance of maintaining monodisperse particle populations in terms of maximizing the predictability of nanoparticle behavior in vivo, the highly monodisperse particle sizes achieved (with no macroaggregate fraction whatsoever apparent over the full range of concentrations tested) is important for fabricating biomedically-relevant microgel particles. The very rapid nature of the fabrication process of the disclosure (only 5 minutes after the PNIPAM-Hzd polymer is pre-heated) offers additional advantages in terms of minimizing processing times and thus particle throughput, particularly relative to other polymerization or emulsion-based techniques.

Increasing the concentration of PNIPAM-Ald in the cross-linking solution results in a small but statistically significant reduction in particle size, as confirmed by both DLS and NTA measurements (Table 1). We attribute the decreased size of the final microgels to the increased efficacy of cross-linking when more PNIPAM-Ald is added, condensing the microgel to a greater degree and thus resulting in lower particle sizes. While the particle sizes measured with DLS and NTA are close in overall size due to the high monodispersity of the particle populations, the average (number-weighted) diameters of the particles with NTA are consistently lower than the average (intensity-weighted) diameters measured by DLS, as would be expected due to the higher weighting of larger particles in DLS. Interestingly, DLS also indicates a significant decrease in polydispersity as the PNIPAM-Ald concentration is increased while NTA does not. Without wishing to be bound by theory, we believe this difference is a result of the differences between the intensity-based and number-based weightings of the DLS and NTA distributions respectively. For DLS, a reduction in the (much more heavily weighted) large particle fraction as the average microgel size is decreased results in a very large decrease in the scattering intensity detected as a result of larger particles; meanwhile, the increased fraction of smaller particles as the overall particle size is reduced is not well-detected by DLS on an intensity-weighted basis. Combined together, these effects result in an effective narrowing of the measured particle size distribution. In contrast, NTA analysis is purely number weighted, such that a reduction in higher size particles is directly offset by an increase in lower size particles to maintain a similar polydispersity.

The electrophoretic mobility of microgels produced using the method of the present disclosure lies in the range of 0.55-0.73×10⁻⁸ m²/Vs for all microgels tested, corresponding to a relatively low zeta potential well below that which is generally considered required for electrostatic stabilization. This result is expected based on the charge neutrality of the polymer building blocks aside from a slight fraction of unfunctionalized acrylic acid residues in PNIPAM-Hzd copolymers. Without wishing to be bound by theory, this result suggests that colloidal stabilization of these microgels is due primarily to a steric mechanism rather than an electrostatic mechanism.

(ii) Effect of Self-Assembly Conditions on Microgel Size

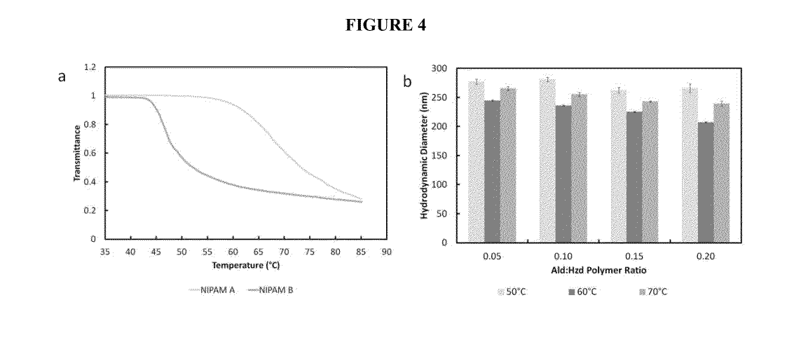

The temperature, mixing time, and stir rate of the self-assembly/cross-linking fabrication procedure were all investigated to identify which factors significantly affect the particle size of the resulting microgel and, by extension, the range over which the microgel size can be tuned by process changes. Temperature is a variable of interest in this respect, as the temperature chosen will help to control the hydrophobicity of each polymer and thus nature of the first aggregation step and/or the penetration of the cross-linking PNIPAM-Ald polymer into the initial nanoaggregate. FIG. 4a shows the lower critical solution temperature behaviors of PNIPAM-Hzd and PNIPAM-Ald measured via UV/vis spectrophotometry, while the changes in microgel particle size achieved by lowering the self-assembly temperature from 70° C. (base case) to 60° C. or 50° C. is shown in FIG. 4b. The LCST of PNIPAM-Hzd at the concentration used for microgel self-assembly was measured to be ˜58° C. (FIG. 4a); as such, at 50° C., the polymer coils are not fully collapsed into precursor particles and the resulting microgel size is larger than observed at 60° C. (at which the PNIPAM-Hzd polymer has begun to collapse according to the LCST result). Further increasing the temperature to 70° C. results in an increase in particle size relative to that observed at 60° C. Without wishing to be bound by theory, we attribute this result to two possible factors: (1) the increased hydrophobicity of the chains at 70° C. (transmittance ˜0.6) relative to 60° C. (transmittance ˜0.9) and (2) the increased diffusion and thus increased collision frequency between polymer chains in solutions. Increased collision frequency between chains with higher hydrophilicities would lead to more polymer on average being incorporated in each nanoaggregate before chain conformation changes can facilitate steric stabilization of the growing microgels. Overall, the data suggest that particle assembly depends on both the magnitude of chain deswelling (and thus the inter-chain polymer affinity) at that temperature (thermodynamics) and the rate of collision of those polymers (kinetics), with the smallest microgels formed just above the LCST at which the polymers are moderately attractive but diffusion is the slowest among any possible attractive condition.

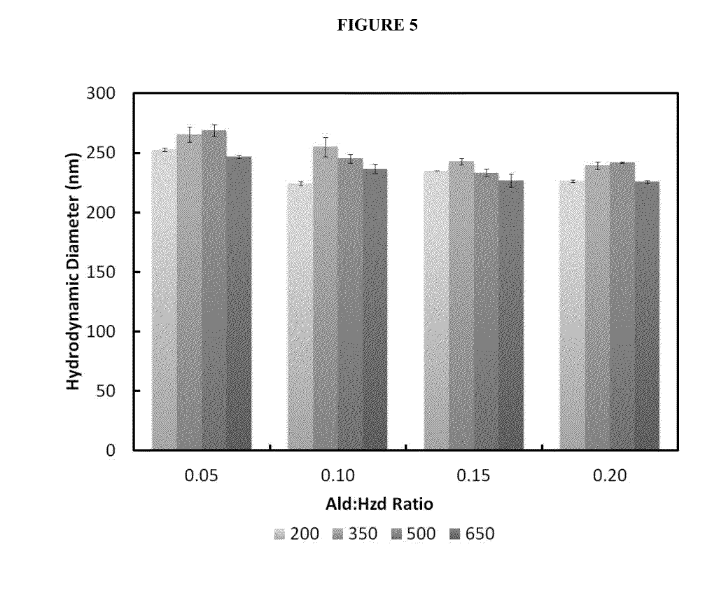

To assess the balance of the thermodynamics and kinetic (collision frequency) contributions to controlling particle size, microgels were produced using different stirring speeds during the PNIPAM-Ald addition process (i.e. changing the kinetics of collisions but not the thermodynamics of inter-chain interactions). Stirring speed did not significantly affect particle size over the 200-650 RPM range studied (FIG. 5). Without wishing to be bound by theory, this result suggests that it is the thermodynamics of the volume phase transition (and its impact in inter-polymer interactions and/or polymer-solvent interactions) rather than the kinetics of changing collision frequencies that primarily drives the particle formation process.

If the opposite sequence (i.e. using PNIPAM-Ald to form the nanoaggregates and then cross-linking with PNIPAM-Hzd) is used to prepare microgels, nanoparticle formation also occurred. A stable suspension of particles was produced at dilute concentrations of PNIPAM-Ald base polymer (<1 mg/mL). This result suggests that the aldehyde-PNIPAM polymers have a lower capacity for sterically stabilizing the precursor nanoaggregates than hydrazide-PNIPAM polymers. This is consistent with the significantly lower LCST observed for PNIPAM-Ald relative to PNIPAM-Hzd (FIG. 3a); that is, PNIPAM-Ald would be more highly condensed and more likely to aggregate to form larger aggregates than the more hydrophilic PNIPAM-Hzd at the same temperature.

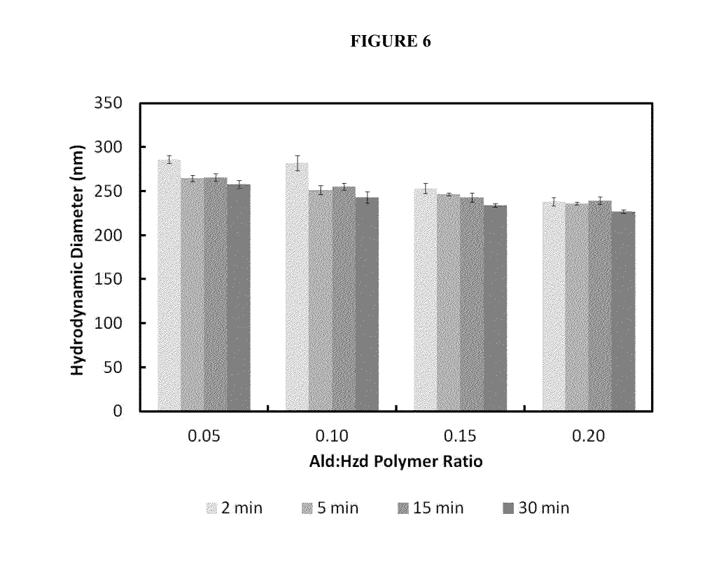

Changing the mixing time between when PNIPAM-Ald was added and when the sample was removed from heating also impacts the particle size, although the differences in this case are relatively small. FIG. 6 shows the hydrodynamic diameters (measured via DLS) of microgels prepared at different Ald:Hzd polymer ratios and left for different periods of time prior to removal of the microgel from the 70° C. heat source. As the mixing time is decreased, larger size microgels were produced across all Ald:Hzd polymer ratios. This is congruent with earlier observations in that less cross-linking (in this case, giving the PNIPAM-Ald polymer less time to do cross-linking) results in less condensation of the initial nanoaggregates and thus larger microgels. Furthermore, as the Ald:Hzd polymer ratio is increased, mixing time has less effect on the particle size. Without wishing to be bound by theory, we hypothesize this observation is related to the rate of cross-linking in each mixture; as the amount of (cross-linking) PNIPAM-Ald added increases, more cross-linking occurs at shorter times, reducing the time required to achieve a plateau value of microgel size (i.e. the time at which cross-linking reaches a maximum due to either or both of steric or stoichiometric limitations). If the reaction is allowed to proceed to longer times (t>45 min), aggregation between particles begins to occur and large precipitates are formed. Again without wishing to be bound by theory, we hypothesize this result is related to consumption of the (hydrophilic) hydrazide groups at the microgel interface which may reduce the potential for steric stabilization; we have previously shown that the hydrazone bond is significantly more hydrophobic than either hydrazide or aldehyde groups and thus lowers the LCST of the near-surface polymer chains that are essential to impart steric stability to the microgels⁷¹.

(iii) Effect of Polymer Concentrations on Microgel Size

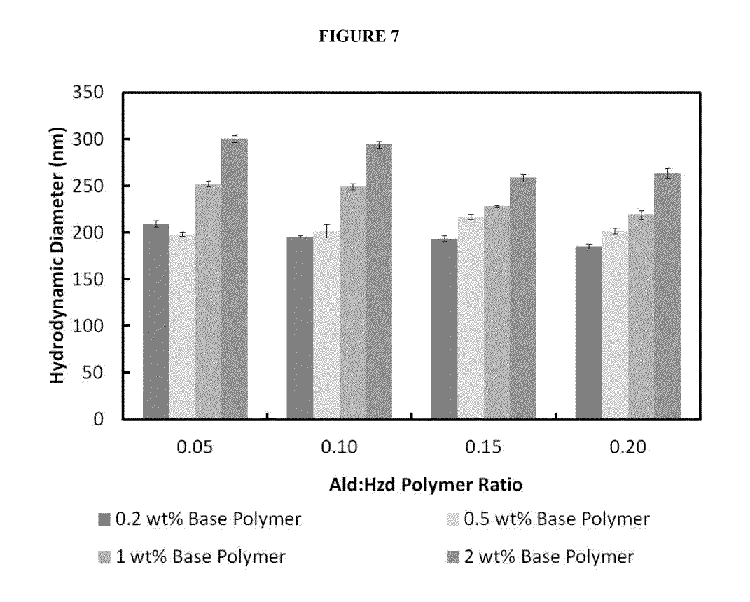

Chemical control over the particle size was also investigated by changing the concentration of PNIPAM-Hzd in the initial seed solution. FIG. 7 shows the hydrodynamic diameter of microgels prepared using different PNIPAM-Hzd initial concentrations. In this case, the Ald:Hzd polymer ratio on the x-axis still represents the ratio of hydrazide:aldehyde functionalized polymer added to form the microgels, with the actual concentration of PNIPAM-Ald added to maintain constant Ald:Hzd ratios changed independent of the PNIPAM-Hzd concentration used. Reducing the concentration of PNIPAM-Hzd in the seed solution decreases the resulting microgel size following PNIPAM-Ald addition. This result is consistent with aggregation kinetics, as polymer solutions with higher concentrations would facilitate an increased probability of collisions between collapsing polymers and existing nuclei, resulting in larger aggregates that would then be cross-linked into larger microgels. A 0.5 w/v % initial solution concentration yielded particle sizes from 200-300 nm depending on the PNIPAM-Ald concentration used for cross-linking. While increasing the Ald:Hzd polymer ratio (i.e. the PNIPAM-Ald concentration) reduces the particle size when 1 w/v % and 2 w/v % PNIPAM-Hzd initial solutions are used (p=0.003 comparing Ald:Hzd=0.05 to Ald:Hzd=0.20), the effect on particle size was lower as a function of Ald:Hzd polymer ratio for the 0.2-0.5 w/v % PNIPAM-Hzd initial solution over the full Ald:Hzd ratio tested (p=0.015). This result suggests that the less cross-linker is required (at least with respect to the inherent steric limitations presented) to fully cross-link the microgels when the seed aggregates are smaller, potentially owing to lower mass transfer limitations to cross-linker diffusion into the seed particles as the diameter of those particles is reduced.

(iv) Scale-Up of Self-Assembly Process

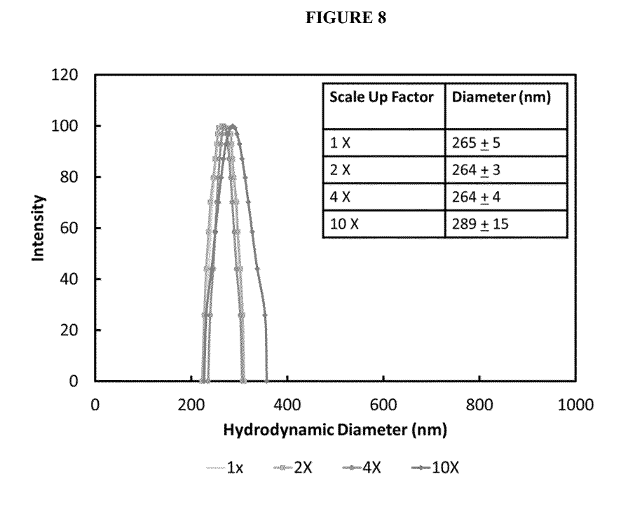

All results discussed to this point were collected using small (5 mL) volume samples that yield relatively low quantities of microgel. For larger-scale testing or ultimate production, effective scale-up of the self-assembly process to larger volumes is required. FIG. 8 shows the particle size distributions of microgels scaled up by total volume by a factor of 2, 4, or 10. No significant increase in hydrodynamic diameter or broadening of the particle size distribution occurs as a function of scaling up the reaction up by a factor of 4. Such a result may be expected if the assembly process is primarily thermodynamically-driven, as factors like local shear during mixing would not significantly impact the final particle properties (indeed, this is consistent with our earlier finding that stir speed does not significantly affect the resulting microgel particle size). At 10 times scale up, there is a small change in particle size. Between the 4× and 10× scale-up trials, the reaction vessels used had to be changed from a 20 mL vial to a 100 mL round-bottom flask; as such, different effects may play a role in governing microgel size such as heat distribution and mixing in the larger volume that did not come into play in the smaller volume. However, given that stable microgels were still produced with highly monodisperse particle populations, this result indicates that this self-assembly method can be directly and successfully scaled to create larger batches as desired.

(v) Microgel Colloidal Stability

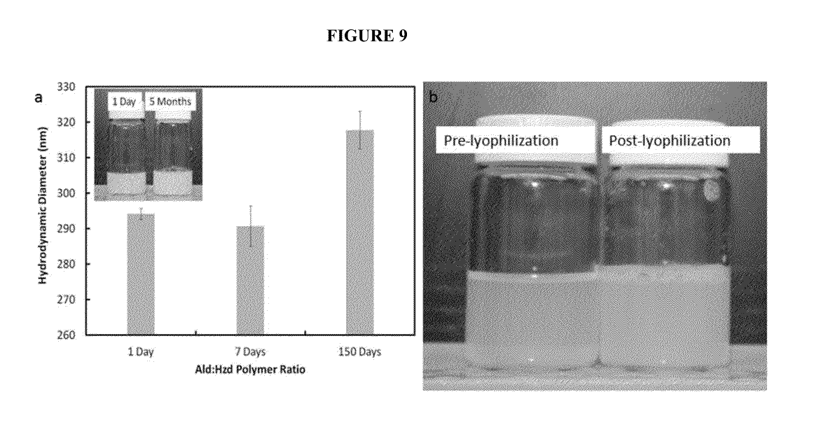

The microgel particles generated by this self-assembly procedure are stable over time; indeed, over a period of at least 5 months, the particles remain in suspension with no visible aggregate present (FIG. 9a). However, there is a net increase in particle size over time as the hydrazone cross-links begin to degrade via hydrolysis, which allows the microgel to subsequently swell; as a result, the particle size increases over time (in the base case microgel, from 287±1 nm after preparation to 317±5 nm after 5 months of incubation). Microgels that were lyophilized following cooling could easily be resuspended in solution (FIG. 9b), with no significant change in particle size observed before and after lyophilization (p=0.4). This result indicates that the microgels produced via this process can be dried and stored without affecting their size or colloidal stability, a significant advantage of this approach relative to other methods of self-assembled microgel production which often produce particles much harder to resuspend or that change size significantly (for example, due to changes in the driving forces for self-assembly as a function of drying) following a drying step⁷⁶.

(vi) Microgel Degradation

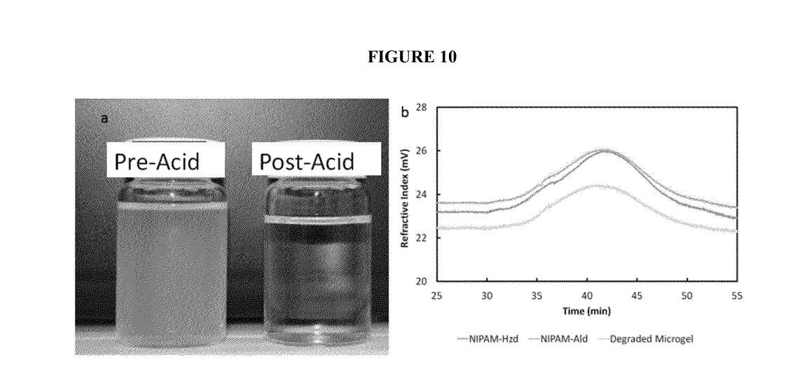

The crosslinking between the hydrazide and aldehyde occurs via hydrazone bond formation, an equilibrium chemistry that is hydrolytically labile (particularly in slight to strong acidic environments, such as the stomach or cell endosomes). To assess the capacity of these microgels to degrade back into the oligomeric building blocks, microgels were exposed to 1M HCl to perform accelerated degradation assays and the degradation products were characterized. After only two hours of exposure, the visual appearance of the microgels solution changed from slightly opaque (indicating microgel presence) to transparent (FIG. 10a), with the count rate observed via DLS reducing 100-fold over this period to a point that no detectable particle size can be measured. Similarly, when the degradation products are analyzed using NTA, no discrete scattering units are imaged at the end of the degradation period. Gel permeation chromatography on the degraded microgel product indicates that the microgels are fully cleaved back into the starting PNIPAM-Ald and PNIPAM-Hzd oligomeric building blocks, with elution times of approximately 40 minutes observed for each precursor polymer (corresponding to molecular weights of <20 kDa, significantly lower than the kidney clearance limit) and the degradation product (FIG. 10b). The capacity for such well-defined degradation based on the assembly of well-defined polymer precursors lies in sharp contrast to microgels prepared with conventional precipitation techniques, in which minimal or no control over the molecular weight of the C—C backbone polymers between cross-links can be exercised^{7,14,77,78}.

(vii) Microgel Phase Transition

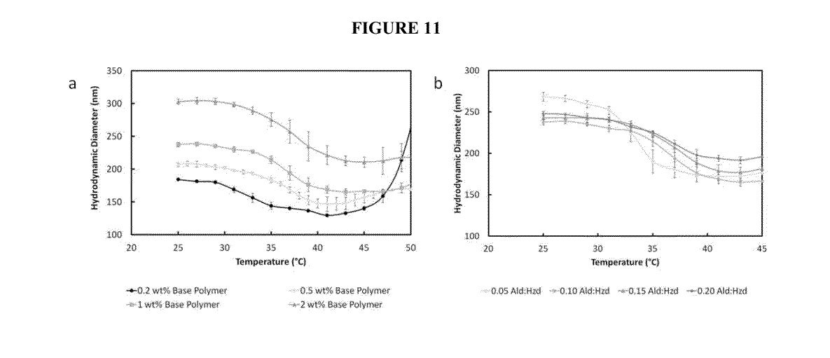

To confirm that the self-assembled microgels exhibit the same thermal phase transition behavior as conventional free radical precipitation-based microgels, particle size was measured as a function of temperature using dynamic light scattering. FIG. 11 a shows the thermal phase transitions of microgels prepared with different PNIPAM-Hzd seed solution concentrations at Ald:Hzd=10, while FIG. 11b shows thermal phase transitions of microgels prepared with a fixed PNIPAM-Hzd seed solution concentration of 1 w/v % but varying Ald:Hzd polymer ratios. The VPTT of most of the microgels tested is slightly higher than observed for conventional precipitation-based microgels, with the temperature at which 50% of the overall diameter change as a function of temperature was observed being ˜38° C. on average based on the DLS measurements. Without wishing to be bound by theory, this slightly increased VPTT is likely attributable to the presence of residual hydrazide groups in the microgel; given that PNIPAM-Hzd has an LCST of ˜58° C. prior to cross-linking, a higher VPTT thus would be expected if even a relatively small fraction of the Hzd groups remain unreacted. However, this slightly higher VPTT is a factor for many biomedical applications of such microgels since thermal collapse occurs just slightly above normal physiological temperature, facilitating (for example) triggered drug release upon encountering of infection sites⁷⁹ or upon external heating of a composite microgel containing magnetic nanoparticles (oscillating magnetic field) or gold nanoshells (near-infrared radiation). PNIPAM-like behavior with VPTT ˜33° C. can also be achieved if desired when smaller Ald:Hzd ratios are used (FIG. 11b). In addition, the breadth of the transition is relatively narrow and highly comparable to conventional microgels, with the onset (5% collapse) and offset (95% collapse) VPTT values lying within no more than a ˜10° C. range for all samples tested.

The magnitude of the phase transition as well as the stability of the collapsed microgels can be observed as a function of the chemistry of each microgel. For microgels with a fixed Ald:Hzd ratio but varying initial concentrations of PNIPAM-Hzd (FIG. 11a), microgels prepared with lower PNIPAM-Hzd concentrations exhibit both lower magnitudes of deswelling and lower colloidal stabilities upon heating (although no sample tested exhibits significant aggregation below ˜45° C., making these materials relevant as reversible thermoresponsive microgels). Without wishing to be bound by theory, this result may be attributed to the smaller size and thus higher specific surface area of microgels prepared at lower PNIPAM-Hzd concentrations, enabling more effective cross-linking with PNIPAM-Ald (i.e. both higher access to the surface and easier penetration into the nanoaggregate) and lower inherent colloidal stability due to the higher interfacial energy of smaller microgel suspensions at a fixed overall mass content. For microgels with a fixed PNIPAM-Hzd concentration but varying Ald:Hzd ratios (FIG. 11b), microgels prepared with lower amounts of PNIPAM-Ald swelled more at low temperature and collapsed more at higher temperature, consistent with the lower total cross-link density that would be expected as the PNIPAM-Ald concentration was decreased. Overall, these results suggest that both the size and the thermosensitivity of the microgels can be tuned over an appreciable range by changing the concentrations of both the seed and cross-linking polymers used to fabricate the microgels.

(viii) Microgel Cytotoxicity

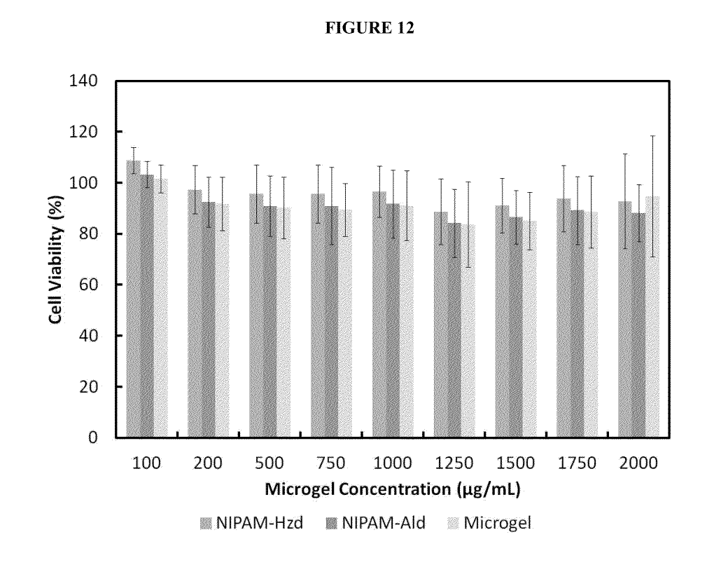

The cytocompatibility of the polymer precursors and the base case microgels was assessed using the MTT metabolic assay and 3T3 mouse myoblast cells. FIG. 12 shows the relative cell viabilities measured with respect to a cell-only control incubated in media only (no materials). No significant cytotoxicity was observed when the cells were exposed to the polymer precursors (also the degradation products, based on FIG. 8b) or base case microgels at concentrations from 0.1 mg/mL to 2 mg/mL. These results are consistent with previous studies involving PNIPAM microgels and PNIPAM containing hydrazide and aldehyde functional groups⁸⁰ and suggest these microgels have potential to be used in vivo without inducing significant cytotoxicity.

### Example 2

### Synthesis of pH Responsive (PNIPAM+DMAEMA) and Glucose Responsive (PNIPAM+PBA) Polymers and Preparation of Anionic and Amphoteric Microgels

Synthesis of Cationic Hydrazide Functionalized PNIPAM (PNIPAM-Hzd-(+)):

Hydrazide functionalized PNIPAM containing 2-dimethylaminoethylmethacrylate (DMAEMA) was prepared as described above but using a backbone polymer containing PNIPAM, acrylic acid, and DMAEMA (the latter incorporated to provide a basic pH-ionizable functional group in the final polymer). The PNIPAM-co-AA-co-DMAEMA backbone polymer was synthesized by dissolving 4.5 g NIPAM, 0.5 g (15 mol % monomer) acrylic acid, and 1.95 g (30 mol % monomer) DMAEMA in 20 mL of absolute ethanol, using the same chain transfer agent and initiator concentrations, polymerization conditions, and ADH grafting conditions described for the neutral PNIPAM-Hzd copolymer. Conductometric titrations (ManTech Inc.) showed that 95 mol % of acrylic acid units were functionalized with hydrazide groups after carbodiimide conjugation. ¹H-NMR showed a total of ˜20 mol % (on a total monomer basis) DMAEMA was incorporated into the polymer.

Synthesis of Acrylated Hydrazide Monomer (tert-butyl 2-acryloylhydrazinecarboxylate, BAHC):

To a solution of boc-carbazate (2.07 g, 15.7 μmoles) in CH₂Cl₂ (75 mL) was added Et₃N (2.40 mL, 17.3 μmoles, 1.1 eq) under a nitrogen atmosphere and the solution was cooled to 0° C. Acryloyl chloride (1.27 mL, 15.7 μmoles) was added dropwise over 5 min and the reaction was allowed to stir at 0° C. for 30 min. The crude reaction mixture was filtered to remove the triethylamine hydrochloride salt and the filtrate was concentrated by rotary evaporation. The product was purified via silica gel column chromatography (2:1->1:1 Hex/EtOAc) to give 1.52 g of the desired product (52% yield). ¹H NMR (600 MHz; DMSO): δ 9.79 (s, 1H), 8.84 (s, 1H), 6.21-6.16 (m, 2H), 5.71 (dd, J=9.2, 3.1, 1H), 1.41 (s, 9H). ¹³C NMR (150 MHz; DMSO): 164.19, 155.20, 129.34, 126.75, 79.17, 28.03.

Synthesis of Anionic Hydrazide Functionalized PNIPAM (PNIPAM-Hzd-(−)):

Anionic hydrazide functionalized PNIPAM was prepared using the same method outlined above for PNIPAM-Hzd-(+) but replacing the 30 mol % of cationic DMAEMA with 30 mol % of BAHC. Following EDC-mediated conjugation of adipic acid dihydrazide to the free acrylic acid groups, the t-butyl protecting group of BAHC was then cleaved off using TFA to expose —COOH groups that are pH ionizable at a defined mole fraction.

Synthesis of Cationic and Anionic Aldehyde Functionalized PNIPAM (PNIPAM-Ald-(+) and PNIPAM-Ald-(−)):

Charged aldehyde-functionalized polymers were prepared using the same protocol as outlined for neutral PNIPAM-Ald but replacing 30 mol % of the NIPAM monomer added with DMAEMA (cationic polymer) or acrylic acid (anionic polymer). Note that NMR analysis confirms that the ester group in DMAEMA is not cleaved to any significant extent during acid-mediated cleavage of the acetal groups of DMEMAm to aldehydes, such that the dual functionalized polymers are generated.

Synthesis of Pheynlboronic Acid (PBA) Functionalized PNIPAM-Hzd (PNIPAM-Hzd-PBA) and PNIPAM-Ald (PNIPAM-Ald-PBA):

Hydrazide-functionalized PNIPAM (PNIPAM-Hzd) was functionalized with PBA using PNIPAM that contained 25 mol % hydrazide groups instead of the 15 mol % hydrazide groups targeted in the non-functionalized polymers as the base polymer, providing 10 mol % hydrazide groups available for PBA functionalization. 0.5 g of PNIPAM-Hzd (25 mol % hydrazides) was dissolved in 200 mL acetate buffer (pH 5) with 12 mg of 4-formylphenylboronic acid (2.1 mol % in the polymer) and 17.2 mg sodium cyanoborohydride to reductively aminate the excess hydrazide groups with PBA. The resulting mixture was mixed for 24 hours, after which it was dialysed against Milli-Q water over six, six-hour cycles (12-14 kDa MWCO) and lyophilized. ¹H-NMR showed a total of 1.3 mol % of monomer residues were functionalized with a PBA group (62% of available PBA attached). A similar reductive amination technique was used to functionalize PNIPAM-Ald with PBA, in this case using 3-aminophenylboronic acid as the grafting molecule. Briefly, 0.5 g of PNIPAM-Ald was dissolved in 200 mL of acetate buffer (pH 5) along with 10 mg of 3-aminophenylboronic acid (9.8 mol %) and 40 mg of sodium cyanoborohydride. The resulting mixture was mixed for 24 hours, after which it was dialysed against Milli-Q water over six, six-hour cycles (12-14 kDa MWCO) and lyophilized. ¹H-NMR showed a total of 8.2 mol % (in total) of PBA was incorporated into PNIPAM-Ald polymer samples (84% yield).

Microgel Characterization:

Microgels particle sizes (dynamic light scattering) and electrophoretic mobilities (PALS) were measured as described previously. The pH response of DMAEMA-functionalized microgels was measured by adjusting pH of the samples prepared as above to the desired value using 1 M HCl or 1M NaOH, while glucose responsiveness of the microgels was assessed by adjusting the glucose concentration of the samples to between 1 mg/mL and 5 mg/mL by adding 1 mL aliquots of concentrated glucose solution to achieve the desired glucose concentration in the microgel solution.

Results & Discussion

(i) Microgel Fabrication

Microgels were formed from PNIPAM-based polymeric precursors functionalized with hydrazide and aldehyde groups. The thermal phase transition of the polymer from coil to aggregate above the LCST is one of the mechanisms driving microgel formation (formation of the PNIPAM-Hzd nanoaggregates that are ultimately cross-linked with PNIPAM-Ald to form microgels). Copolymerization of 2-dimethylaminoethylmethacrylate into the PNIPAM-Hzd polymer backbone provided monomer units that were cationic at physiological pH (the pKₐ of DMAEMA is ˜8.0) but neutral at higher pH values, rendering the microgel pH-responsive. Microgels were produced at pH ˜7.0, meaning that microgels were slightly cationic under preparation conditions and swell during production and after being stabilized by the formation of the hydrazone bond following PNIPAM-Ald addition. Analogously, glucose-responsive PBA-containing microgels were produced by grafting 4-formylphenylboronic acid to PNIPAM-Hzd and/or 3-aminophenylboronic acid to PNIPAM-Ald, with microgels produced using the same precipitation/cross-linking method. All functional microgels were created using hydrazide-functionalized PNIPAM solution concentrations of 0.5 w/v % as opposed to the 1 w/v % concentration used for fabricating neutral microgels.

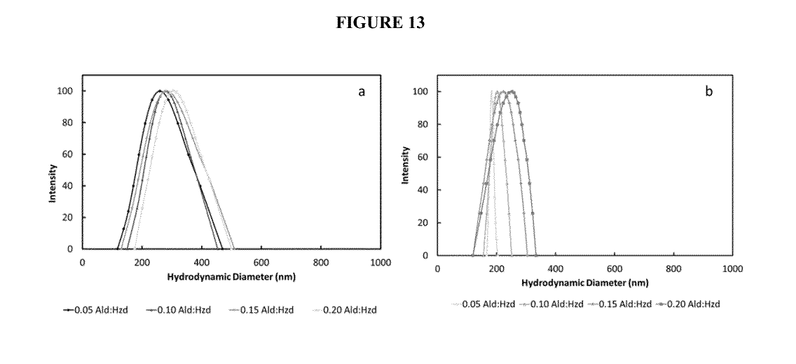

After addition of the aldehyde polymer, the microgels remain stable at temperatures both above and below the VPTT and do not dissociate back into the starting polymers upon cooling. FIG. 13 shows the size distributions of the microgels (from DLS measurements) for both PBA-functionalized and DMAEMA-functionalized PNIPAM microgels produced using our self-assembly technique, while Table 2 shows the mean particle sizes and polydispersities of the functionalized microgels produced.

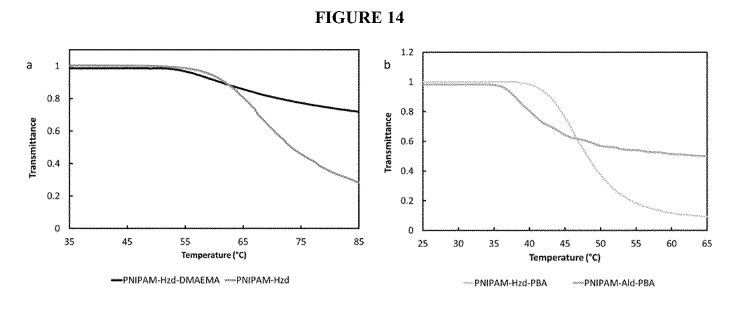

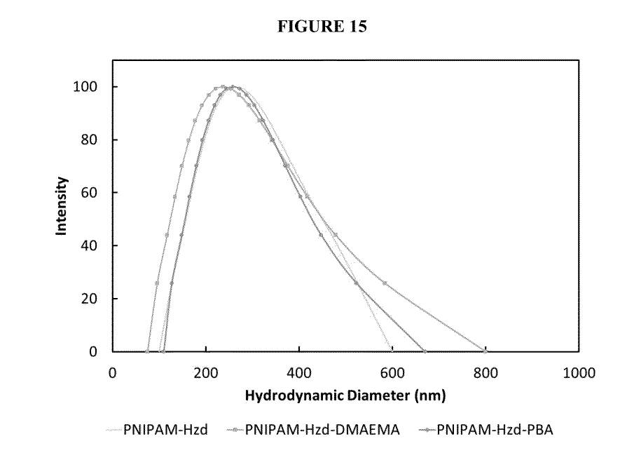

All microgels exhibit a single population DLS peak with relatively narrow polydispersity regardless of concentration of the crosslinker polymer added to the production pot (note that a generated polydispersity value of <0.1 is generally interpreted as a monodisperse population in DLS⁸¹). This polydispersity is especially narrow relative to that typically achieved using inverse emulsion techniques that are commonly used as alternative strategies to form degradable microgels⁸². Since maintaining monodisperse microgel populations is expected to lead to more predictable behavior in vivo⁸³, the relatively low monodispersity of these functionalized microgels is an important aspect of these microgels in biomedical applications. DMAEMA-functionalized microgels exhibit significantly higher particle sizes relative to non-functionalized and PBA-functionalized microgels, a result that can likely be attributed to the presence of a positive charge provided by the DMAEMA monomer units during the aggregation step, which both limits the degree of chain collapse that occurs under the 70° C. preparation conditions (FIG. 14a) and induces swelling of the microgels under the pH (˜7) measurement conditions. Given that the aggregate size distributions between PNIPAM-Hzd and PNIPAM-DMAEMA-Hzd are similar at 70° C. (FIG. 15), this suggests that the chain density inside the aggregates in each case may be different, with PNIPAM-Hzd likely containing a denser aggregate core than PNIPAM-DMAEMA-Hzd. However, both PNIPAM-Hzd and PNIPAM-DMAEMA-Hzd exhibit similar LCSTs (FIG. 14a).

As the amount of PNIPAM-Ald used to produce DMEAMA-functionalized microgels is increased, an increase (0.05 to 0.20 Ald:Hzd ratio, p=0.025) in particle size is observed. This effect may be due to the increased hydrophobicity of the nanoparticle as a function of PNIPAM-Ald cross-linking (and thus consumption of the highly hydrophilic hydrazide groups to form significantly less hydrophilic hydrazone bonds), potentially resulting in additional nanoaggregation on the same time scale of the cross-linking reactions to result in larger particles.

The sizes of PBA-functionalized microgels are smaller than microgels prepared with PNIPAM-Hzd or PNIPAM-Hzd-DMAEMA. The pKₐ of PBA groups is ˜9, although the secondary amine linkage used to attach the phenylboronic acid group to the backbone polymer does significantly reduce that pKₐ to close to physiological range⁸⁴. However, a significant fraction of PBA groups (at least in the absence of glucose) remain unprotonated and thus relatively hydrophobic at pH 7.4. As a result, functionalization of PBA groups results in lowering of the LCST to ˜41° C. (significantly lower than PNIPAM-Hzd, FIG. 14b), with the total transmittance of the solution decreasing significantly (FIG. 14b). This result is indicative of increased chain compaction via hydrophobic self-association of PBA groups which reduces the size of the resulting formed microgels.

When the amount of PNIPAM-Ald polymer used to prepare the microgels is increased, the microgel size also increases, similar to that observed for DMEAMA-functionalized microgels but again in contrast to the non-functionalized microgels. We attribute this result to one of two possible effects. First, since the PNIPAM-Hzd-PBA nanoaggregates appear to be highly collapsed based on FIG. 14b, the penetration of PNIPAM-Ald into the microgel is likely to be low; as such, adding additional aldehyde polymer may instead create a “hairy” surface layer on the microgel as opposed to effectively increase the cross-link density of the microgel, resulting in a larger microgel hydrodynamic diameter (and potentially a more sterically-stabilized microgel). Second, increased covalent bond formation upon higher PNIPAM-Aid addition would reduce the network mobility and thus potentially reduce the capacity for hydrophobic interactions between PBA groups that can function as physical cross-links; we have previously observed similar effects in bulk hydrogels that contain hydrophobic self-association domains⁸⁵. As a result, while the covalent cross-link density of the microgel would increase as more PNIPAM-Ald is added, the overall (physical+covalent) cross-link density may effectively be reduced, leading to additional microgel swelling.

(ii) Effects of pH Change of DMAEMA Functionalized Microgel

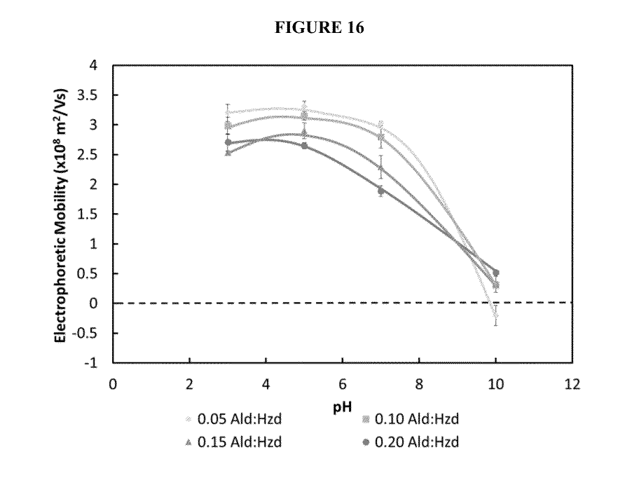

The presence of DMAEMA within the microgels allows them to become responsive to changes in pH though the protonation of the amino group at pH values below the pKₐ of DMAEMA (˜8). FIG. 16 shows the electrophoretic mobility of the microgels as a function of pH.

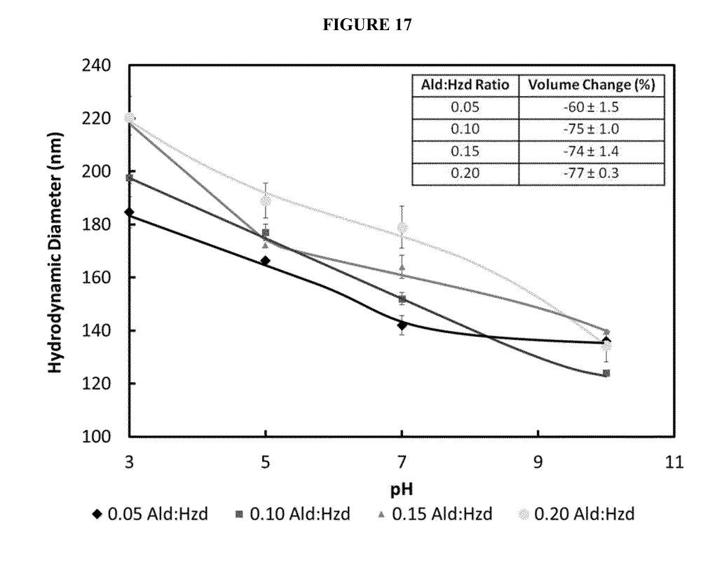

All DMAEMA-functionalized microgels exhibit a large cationic charge at lower pH which is reduced to nearly zero for all microgels tested as the pH is increased above the pKa to pH ˜10. This change in the charge content of the microgel is directly associated with a deswelling of the microgel as the pH is increased, as observed in FIG. 17.

As the pH of the solution is increased, deprotonation of the charged cationic DMAEMA groups results in a reduction in Donnan equilibrium contributions to microgel swelling and thus a decrease in microgel size, resulting in microgels that are ˜70% smaller than when fully swollen. The magnitude of deswelling observed as the pH is increased is similar for all microgel cross-linking densities explored, with deswelling occurring roughly equivalently at each pH value tested for each microgel given that the PNIPAM-Hzd-DMAEMA polymer (which alone governs the pH-responsiveness of the final microgel) is present at the same concentration in each microgel tested.

Note that the colloidal stability of DMAEMA-functionalized microgel is high; microgels remained in suspension over the course of 2 weeks at physiological pH without exhibiting any sign of aggregation and only minimal changes in particle size (associated with slow degradation of the hydrazone cross-links, as observed earlier for the non-functionalized PNIPAM microgels).

(iii) Effects of Glucose Concentration on PBA-Functionalized Microgel Swelling

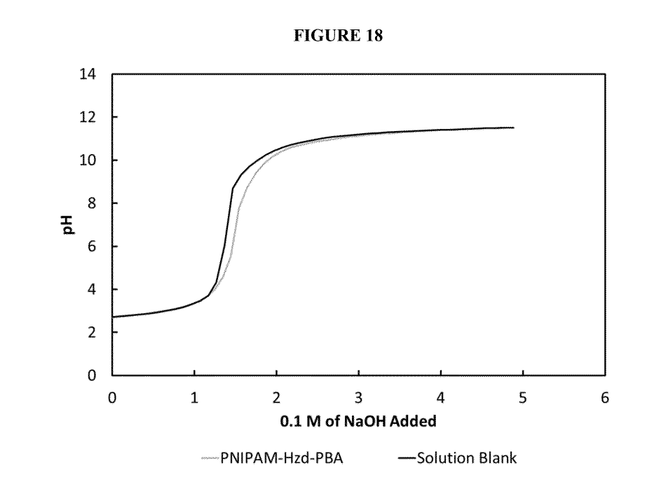

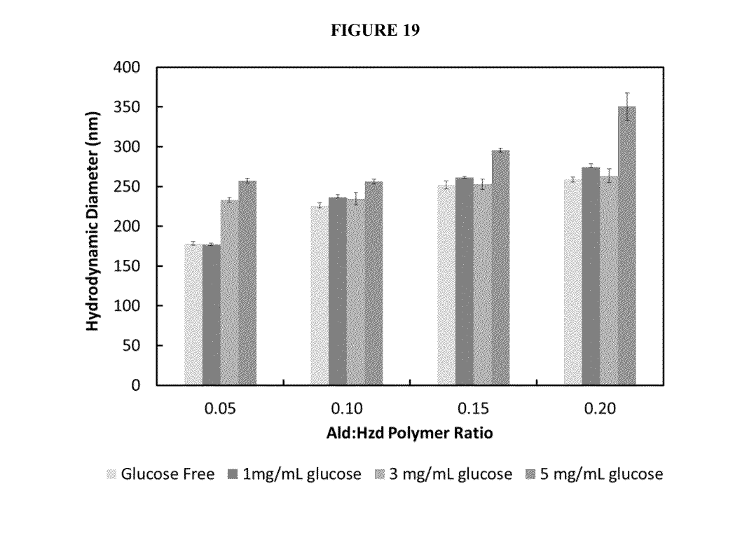

Titration analysis indicated that ionization of the secondary amine-linked PBA groups occurs at pH values at and even below physiological pH (FIG. 18), such that glucose-responsive swelling would be expected at pH 7.4. The glucose responsive swelling of the PBA-functionalized microgels was assessed at room temperature in aqueous solutions at glucose concentrations up to 5 mg/mL. The change in microgel size as a function of solution glucose concentration is shown in FIG. 19.

Note that as more PNIPAM-Ald is added, an increased amount of PBA is incorporated into the microgel since the PNIPAM-Ald used contains 8.2 mol % PBA groups (in contrast, the PNIPAM-Ald used in the DMAEMA study did not contain any additional DMAEMA residues). At all microgel formulations, there is no significant response (0.05, 0.10, 0.15 Ald:Hzd) or only a small (albeit statistically significant) response (0.20 Ald:Hzd) to the addition of glucose at low solution concentrations (1 mg/mL). As the glucose concentration increases to 3 mg/mL, microgels containing less PBA (in particular, the 0.05 Ald:Hzd microgel) begin to swell. As the concentration of glucose in the microgel environment is increased further (5 mg/mL), all microgel formulations experience a significant increase in swelling. This result can be interpreted in two ways. First, glucose contains two cis-diol groups and thus can in theory form a bis-bidentate complex with PBA residues⁴⁸. Thus, at lower concentrations at which glucose is less likely to fully saturate all PBA binding sites, the probability of glucose-induced cross-linking is relatively higher, restricting any swelling response that may be observed as a result of the shift in boronate ester ionization observed upon glucose binding. As glucose concentrations are increased, single PBA-glucose interactions become more likely and thus swelling due to boronate ionization is significantly enhanced. Second, a critical concentration of glucose may exist (depending on the PBA content of the microgel) at which an equilibrium shift occurs in the direction of forming more conjugates, leading to a significant increase in localized charge density and microgel hydrophilicity that causes the microgels to swell⁶². This is consistent with other protonation/deprotonation phase transitions, at which a critical charge content is required in order to result in a significant change in microgel swelling⁸⁷. As such, careful manipulation of PBA content can result in different glucose responses and degrees of swelling to meet specific application requirements.

(iv) Microgel Phase Transition

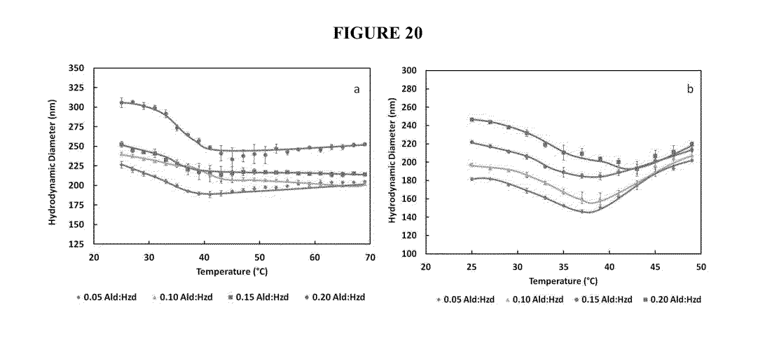

To ensure that the self-assembled functionalized microgels still maintain a thermal phase transition, particle size was measured as a function of temperature. FIG. 20a displays the thermal phase transitions of microgels functionalized with DMAEMA whereas FIG. 20b shows thermal phase transitions of microgels functionalized with PBA groups.

The VPTT of the microgels is similar to that observed for conventional monomer, free radical initiated based NIPAM microgels, with 50% of the overall diameter change observed over the full phase transition occurring at ˜37-38° C. on average based on the DLS measurements for all microgels tested. The fact that the microgels collapse just above physiological temperature makes these attractive for biomedical applications, facilitating (for example) triggered release of metabolites or active pharmaceutical ingredients at sites of infection⁸⁸. The breadth of the transition is also relatively narrow, with onset (5% collapse) and offset (95% collapse) VPTT occurring within a ˜10° C. range for all samples tested. DMAEMA-functionalized microgels remain stable upon heating except for microgels prepared at the lowest Ald:Hzd ratio, which exhibit slight re-aggregation above the VPTT. In contrast, PBA-functionalized microgels all aggregate to some extent above the VPTT, although the temperature at which this aggregation occurs increases systematically as the Ald:Hzd ratio is increased. It is hypothesized that this observation is attributable to the higher covalent cross-link density achieved at higher Ald:Hzd ratios, making hydrophobic interactions between uncharged PBA groups less likely to occur and reducing the likelihood of aggregation between collapsed particles via such interactions.

(vi) Anionic Microgels

Analogously to the cationic (DMEAMA) microgels, anionic microgels can be formed using acid-functionalized precursor polymers. Significantly different particle sizes and polydispersities are achieved depending on the type of anionic polymer used. Table 3 shows the sizes and polydispersities of microgels generated by (1) use of PNIPAM-Hzd-(−) as the seed polymer and PNIPAM-Ald as the cross-linking polymer, (2) use of PNIPAM-Hzd as the seed polymer and PNIPAM-Ald-(−) as the cross-linking polymer, or (3) use of PNIPAM-Hzd-(−) as the seed polymer and PNIPAM-Ald-(−) as the cross-linking polymer.

Independent of the Ald:Hzd ratio used to make the microgels, samples prepared with either an anionic seed (Hzd-functionalized, −/0) or an anionic cross-linker (Ald-functionalized, 0/−) polymer exhibited consistent particle sizes and relatively low (˜0.20) polydispersities, with sizes decreasing as the amount of cross-linking Ald-functionalized polymer added is increased. However, the polydispersities are in all cases higher than that achieved with the all neutral polymers. Of note, if the anionic polymer is used as the cross-linker, the size of the particles is significantly higher than that achieved if the anionic polymer is used as the seed and is roughly double the particle size achieved if a neutral PNIPAM-Ald cross-linking polymer is used. This is consistent with shell-localized functional groups and suggests that the sequence of addition can be used to engineer the functional group distribution inside these microgels, of significant use for achieving tunable phase transition behaviours in microgels. If both the seed and cross-linking polymers are anionic (−/−), very poor control over particle size is achieved, accompanied by broad polydispersities. Thus, the presence of one neutral polymer appears to be important for achieving monodisperse particles.

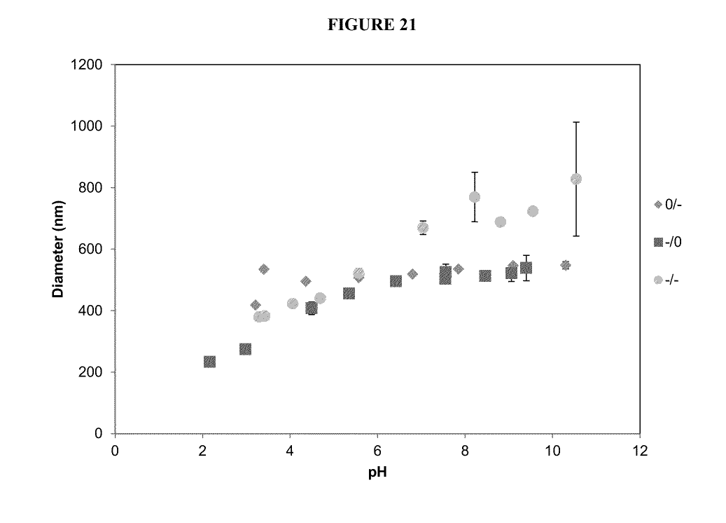

The particle size pH response curves associated with each sequence of addition are shown in FIG. 21.

Since 10-fold more (by mass) hydrazide-functionalized (seed) polymer is used relative to aldehyde-functionalized (cross-linking) polymer, microgels in which PNIPAM-Hzd-(−) is used as the core have significantly more pH-responsive functional group content than microgels in which only the cross-linking PNIPAM-Ald-(−) polymer is used to introduce charge. Thus, the (−/0) microgel (PNIPAM-Hzd-(−) seed, neutral cross-linker) swells significantly more as a function of pH than the (0/−) microgel (neutral seed, PNIPAM-Ald-(−) cross-linker), with roughly 10-fold changes in volume achievable upon —COOH ionization. When anionic groups are present on both polymers (−,−), a higher swelling ratio is achieved, but at a cost of monodispersity. Thus, the results indicate that (1) pH-responsive swelling of acid-functionalized microgels can be achieved (2) the distribution of those functional groups can be influenced by the sequence of anionic pre-polymer addition and (3) the use of neutral PNIPAM-Ald or PNIPAM-Hzd as either the seed or the cross-linker is maintains narrow polydispersities in the final product.

(vii) Amphoteric Microgels

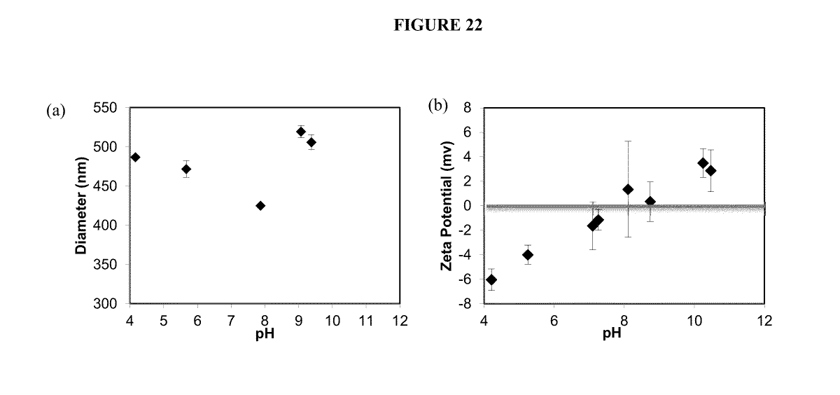

Similarly, by using one precursor polymer that is anionic and one that is cationic, amphoteric microgels can be produced. FIG. 22 shows the particle size and zeta potential of an amphoteric microgel prepared using PNIPAM-Hzd-(+) as the seed and PNIPAM-Ald-(−) as the cross-linker.

The microgel exhibits classical amphoteric microgel behaviour, with a swelling minimum observed at pH ˜8 (FIG. 22a) corresponding to the isoelectric point of the microgel where the number of cationic and anionic charges are equally balanced (FIG. 22b). Swelling is observed above or below this value, corresponding to higher net cationic or anionic charges. Amphoteric microgels of this type are of significant potential interest in biomedical applications given that amphoteric microgels are noted to have particularly low protein adsorption and the potential to strongly bind proteins, which are themselves amphoteric, to achieve extended protein delivery.

### Example 3

### Self-Assembly Method of PNIPAM Applied to Other Materials (POEGMA)

Synthesis of Hydrazide Functionalized poly(oligoethylene glycol methacrylate) (POEGMA-Hzd):

POEGMA-Hzd was prepared by adding AIBMe (37 mg, 0.14 mmol), M(EO)₂MA (3.5 g, 18.6 mmol), OEGMA₄₇₅ (0.50 g, 1.1 mmol), AA (0.60 g, 8.3 mmol) and TGA (7.5 uL, 0.12 mmol) to a 100 ml Schlenk flask. Dioxane (20 mL) was added and the solution was purged with nitrogen for at least 30 min. The 9:1 molar ratio of M(EO)₂MA:OEGMA₄₇₅ was selected to achieve a thermal phase transition at ˜32° C., as per data reported for bulk hydrogels⁸⁹. Subsequently, the flask was sealed and submerged in a pre-heated oil bath at 75° C. for 4 h under magnetic stirring. After removing the solvent, the resulting poly(ethylene glycol methacrylate-co-acrylic acid) polymer was purified by dialysis against DIW for a minimum of six (6+h) cycles and lyophilized to dryness. The carboxylic acid groups on the acrylic acid residues were subsequently converted to hydrazide groups via a carbodiimide-mediated conjugation using a fivefold molar excess of adipic acid dihydrazide. The polymer (3.0 g) was dissolved in 150 mL DIW in a 500 ml round-bottom flask. ADH (3.6 g, 20.5 mmol) was added and the pH of the solution was adjusted to pH=4.75 using 0.1 M HCl. Subsequently, EDC (1.6 g, 10.2 mmol) was added and the pH maintained at pH=4.75 by the dropwise addition (as required) of 0.1M HCl over 4 h. The solution was left to stir overnight, dialysed against DIW for a minimum of six (6+h) cycles and lyophilized. The degree of functionalization was determined from conductometric base-into-acid titration (ManTech Associates) using 50 mg of polymer dissolved in 50 ml of 1 mM NaCl as the analysis sample and 0.1 M NaOH as the titrant. The polymers were stored as 20 w/w % solutions in PBS at 4° C.

Synthesis of Aldehyde Functionalized poly(oligoethylene glycol methacrylate) (POEGMA-Ald):

POEGMA-Ald was prepared by adding AIBMe (32 mg, 0.14 mmol), M(EO)₂MA (3.10 g, 16.5 mmol), OEGMA₄₇₅ (0.90 g, 1.9 mmol), N-(2,2-dimethoxyethyl)methacrylamide (DMEMAm; 1.30 g, 7.5 mmol) and TGA (7.5 uL, 0.12 mmol) to a 100 ml Schlenk flask. The same OEGMA monomer ratio was used to ensure both precursor polymers remain thermosensitive and thus capable of undergoing thermally-driven self-assembly. Dioxane (20 mL) was added and the solution purged with nitrogen for at least 30 min. Subsequently, the flask was sealed and submerged in a pre-heated oil bath at 75° C. for 4 h under magnetic stirring. After, it was purified by dialysis against DIW for a minimum of six (6+h) cycles and lyophilized to dryness. The acetal groups of PO₁₀A were subsequently converted to aldehydes by dissolving 3.5 g of the copolymer prepared above in 50 mL DIW and 50 mL 1.0 M HCl in a 500 ml round-bottom flask. The solution was left to stir for 24 h, dialysed for a minimum of six (6+h) cycles and lyophilized to dryness. The polymer was stored as 20 w/w % solution in PBS at 4° C.

Results and Discussion

A self-assembly method identical to that described for PNIPAM was used to form microgels. Initially the seed polymer (either POEGMA-Ald or POEGMA-Hzd, 5 or 10 mg/mL) was heated in solution. Subsequently, the cross-linking polymer is added dropwise to the seed solution in ratios of 5, 10, 15 or 25% to the total seed polymer. Characterization of the microgels was carried out using dynamic light scattering (DLS) to determine the microgel diameter. Average diameters for different preparation conditions are reported in Table 4. As shown, while some preparations yielded larger polydispersities (particularly when self-assembly was conducted at temperatures at or below the LCST values of one or more of the precursor polymers, as indicated also in Table 4), preparations conducted just above the volume phase transition temperature of the seed polymer solution appear to give the most consistent particle sizes with the smallest polydispersities. In particular, highly monodisperse (polydispersity <0.1) particles are formed using a 5 mg/mL POEGMA-Hzd seed solution and POEGMA-Ald as the cross-linker with cross-linking conducted at 67° C. (LCST+5° C. of seed polymer), with the particle size tunable over a >100 nm range based on the amount of POEGMA-Ald added may suffer from significantly fewer translational barriers than faced by PNIPAM-based microgels. Thus, monodisperse microgels based on POEGMA can be prepared using the same self-assembly method used for PNIPAM.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1
Size and polydispersity of microgels produced at different
Ald:Hzd ratios as measured via dynamic light scattering
(DLS) and nanoparticle tracking analysis (NTA).

| | | | DLS | NTA |
|---|---|---|---|---|
| | | | Polydis- | Polydis- |
| Microgel | DLS Size | NTA Size | persity^{a} | persity^{b} |
| 0.05 | 252.2 + 3.3 | 206.5 + 4.9 | 0.094 + 0.022 | 1.13 + 0.02 |
| Ald:Hzd | | | | |
| 0.10 | 249.0 + 3.3 | 203.4 + 1.7 | 0.107 + 0.009 | 1.11 + 0.02 |
| Ald:Hzd | | | | |
| 0.15 | 228.0 + 1.0 | 200.3 + 3.2 | 0.078 + 0.028 | 1.12 + 0.01 |
| Ald:Hzd | | | | |
| 0.20 | 218.9 + 4.5 | 196.1 + 6.9 | 0.09 + 0.020 | 1.10 + 0.1 |
| Ald:Hzd | | | | |

^{a}Measured via Brookhaven polydispersity algorithm on intensity-weighted raw data;
^{b}Calculated as D_{w}/Dₙ based on the number-weighted size distribution measured by NTA

TABLE 2
Size and polydispersity of microgels produced at different Ald:Hzd
ratios as measured via dynamic light scattering (DLS) at 25° C.

| | DLS Size | DLS |
|---|---|---|
| Microgel | (nm) | Polydispersity |
| 0.05 Ald:Hzd - DMAEMA | 262.1 ± 4.2 | 0.11 ± 0.02 |
| 0.10 Ald:Hzd - DMAEMA | 280.8 ± 4.0 | 0.12 ± 0.03 |
| 0.15 Ald:Hzd - DMAEMA | 287.4 ± 4.8 | 0.13 ± 0.03 |
| 0.20 Ald:Hzd - DMAEMA | 312.9 ± 9.0 | 0.13 ± 0.05 |
| 0.05 Ald:Hzd - PBA | 178.4 ± 2.2 | 0.08 ± 0.03 |
| 0.10 Ald:Hzd - PBA | 201.1 ± 3.8 | 0.10 ± 0.04 |
| 0.15 Ald:Hzd - PBA | 222.9 ± 3.2 | 0.10 ± 0.03 |
| 0.20 Ald:Hzd - PBA | 251.2 ± 4.9 | 0.11 ± 0.03 |

TABLE 3
Particle sizes (from dynamic light scattering) and polydispersities of
microgels produced by sequential addition of PNIPAM-Hzd (charged or uncharged)
and PNIPAM-Ald (charged or uncharged).

| | PNIPAM-Hzd-(−) + | | PNIPAM-Hzd + | | PNIPAM-Hzd-(−) + | |
|---|---|---|---|---|---|---|
| | PNIPAM-Ald (−/0) | | PNIPAM-Ald-(−) (0/−) | | PNIPAM-Ald-(−) (−/−) | |
| Ald:Hzd | Diameter | | Diameter | | Diameter | |
| Ratio | (nm) | Polydispersity | (nm) | Polydispersity | (nm) | Polydispersity |
| 0.05 | 282 ± 13 | 0.21 ± 0.01 | 448 ± 7 | 0.18 ± 0.04 | 496 ± 76 | 0.31 ± 0.01 |
| 0.10 | 278 ± 1 | 0.20 ± 0.01 | 421 ± 5 | 0.20 ± 0.03 | 403 ± 10 | 0.29 ± 0.01 |
| 0.15 | 265 ± 5 | 0.24 ± 0.02 | 410 ± 6 | 0.19 ± 0.02 | 672 ± 158 | 0.35 ± 0.03 |
| 0.20 | 263 ± 6 | 0.22 ± 0.01 | 401 ± 6 | 0.18 ± 0.03 | 467 ± 62 | 0.35 ± 0.02 |

TABLE 4
Average diameter and polydispersity of individual microgel batches with different
core polymers, core concentrations and crosslink ratios at different temperatures.

| | | | POEGMA-Hzd crosslinker | | | | POEGMA-Ald crosslinker | | | |
|---|---|---|---|---|---|---|---|---|---|---|
| | | | 5 mg/mL | | 10 mg/mL | | 5 mg/mL | | 10 mg/mL | |
| | | | POEGMA-Ald | | POEGMA-Ald | | POEGMA-Hzd | | POEGMA- | |
| | | | seed | | seed | | seed | | Hzd seed | |
| | T | Xlink | Avg | | Avg | | Avg | | Avg | |
| | (° C.) | Ratio | (nm) | PD | (nm) | PD | (nm) | PD | (nm) | PD |
| | 72 | 5 | 192.2 | 0.38 | 321.4 | 0.24 | 309 | 0.16 | 284.1 | 0.13 |
| | | 10 | 138.6 | 0.36 | 365.9 | 0.25 | | | 273.8 | 0.13 |
| | | 15 | 128 | 0.33 | 401.5 | 0.28 | | | 256.8 | 0.13 |
| | | 20 | 131.6 | 0.32 | 356.1 | 0.29 | 445 | 0.18 | 282.3 | 0.14 |
| | 67 | 5 | 53.2 | 0.21 | 53.2 | 0.21 | 251.9 | 0.087 | Samples | |
| | | 10 | 57.5 | 0.2 | 112 | 0.32 | 208.1 | 0.1 | aggregated | |
| | | 15 | 59 | 0.23 | 128.5 | 0.32 | 175.4 | 0.12 | on multiple | |
| | | 20 | 67.5 | 0.26 | 124.1 | 0.32 | 162.8 | 0.1 | attempts | |
| LCST A | 62 | 5 | 114.9 | 0.36 | 93.3 | 0.31 | 179.3 | 0.36 | 400.3 | 0.12 |
| polymer | | 10 | 51.3 | 0.24 | 101 | 0.32 | 132.4 | 0.33 | 306.8 | 0.11 |
| MS-1-44 | | 15 | 49.8 | 0.24 | 114.4 | 0.31 | 113.1 | 0.3 | 241.4 | 0.1 |
| | | 20 | 53.8 | 0.23 | 112 | 0.3 | 109.3 | 0.29 | 202.9 | 0.12 |
| LCST B | 57 | 5 | 94.6 | 0.26 | 74.6 | 0.29 | 441.3 | 0.35 | 351.7 | 0.32 |
| polymer | | 10 | 91.9 | 0.26 | 83.8 | 0.32 | 406.7 | 0.29 | 208.2 | 0.32 |
| MS-1-45 | | 15 | 90.1 | 0.25 | 88.5 | 0.32 | 440 | 0.26 | 182.2 | 0.31 |
| | | 20 | 102.9 | 0.26 | 88.8 | 0.32 | 761 | 0.39 | 189.6 | 0.33 |

### REFERENCES

(1) Morris, G. E.; Vincent, B.; Snowden, M. J. J. Colloid Interface Sci. 1997, 190, 198.
(2) Parasuraman, D.; Sarker, A.; Serpe, M. Colloid. Polym. Sci. 2013, 291, 1795.
(3) Gichinga, M. G.; Striegler, S.; Dunaway, N. A.; Barnett, J. D. Polymer 2010, 51, 606.
(4) Wang, Q.; Libera, M. Colloids Surf, B 2014, 118, 202.
(5) Hoare, T.; Pelton, R. Langmuir 2008, 24, 1005.
(6) Zhou, T.; Wu, W.; Zhou, S. Polymer 2010, 51, 3926.
(7) Zhang, X.; Lu, S.; Gao, C.; Chen, C.; Zhang, X.; Liu, M. Nanoscale 2013, 5, 6498.
(8) Bitar, A.; Fessi, H.; Elaissari, A. J. Biomed. Nanotechnol. 2012, 8, 709.
(9) Wang, Y.; Nie, J.; Chang, B.; Sun, Y.; Yang, W. Biomacromolecules 2013, 14, 3034.
(10) Balaceanu, A.; Mayorga, V.; Lin, W.; Schürings, M.-P.; Demco, D.; Baer, A.; Winnik, M.; Pich, A. Colloid. Polym. Sci. 2013, 291, 21.
(11) Hoare, T.; Pelton, R. Biomacromolecules 2008, 9, 733.
(12) Hu, X.; Tong, Z.; Lyon, L. A. J. Am. Chem. Soc. 2010, 132, 11470.
(13) Cai, T.; Marquez, M.; Hu, Z. Langmuir 2007, 23, 8663.
(14) Ulasan, M.; Yavuz, E.; Bagriacik, E. U.; Cengeloglu, Y.; Yavuz, M. S. Journal of Biomedical Materials Research Part A 2014, n/a.
(15) Hu, Z.; Cai, T.; Chi, C. Soft Matter 2010, 6, 2115.
(16) Scott, E. A.; Nichols, M. D.; Cordova, L. H.; George, B. J.; Jun, Y.-S.; Elbert, D. L. Biomaterials 2008, 29, 4481.
(17) Oh, J. K.; Siegwart, D. J.; Lee, H.-i.; Sherwood, G.; Peteanu, L.; Hollinger, J. O.; Kataoka, K.; Matyjaszewski, K. J. Am. Chem. Soc. 2007, 129, 5939.
(18) Agrawal, G.; Wang, J.; Bruster, B.; Zhu, X.; Moller, M.; Pich, A. Soft Matter 2013, 9, 5380.
(19) Binauld, S.; Stenzel, M. H. Chem. Commun. 2013, 49, 2082.
(20) Ghugare, S. V.; Chiessi, E.; Sakai, V. G.; Telling, M. T. F.; Wadgaonkar, P. P.; Paradossi, G. Macromol. Symp. 2013, 329, 27.
(21) Aguirre, G.; Ramos, J.; Forcada, J. Soft Matter 2013, 9, 261.
(22) Chan, N.; An, S. Y.; Yee, N.; Oh, J. K. Macromol. Rapid Commun 2014, 35, 752.
(23) Zhang, Q.; Re Ko, N.; Kwon Oh, J. RSC Advances 2012, 2, 8079.
(24) Aleksanian, S.; Khorsand, B.; Schmidt, R.; Oh, J. K. Polymer Chemistry 2012, 3, 2138.
(25) Morimoto, N.; Qiu, X.-P.; Winnik, F. M.; Akiyoshi, K. Macromolecules 2008, 41, 5985.
(26) Vinogradov, S. V.; Bronich, T. K.; Kabanov, A. V. Adv. Drug Del. Rev. 2002, 54, 135.
(27) Urakami, H.; Hentschel, J.; Seetho, K.; Zeng, H.; Chawla, K.; Guan, Z. Biomacromolecules 2013, 14, 3682.
(28) Rogers, K. R.; Bradham, K.; Tolaymat, T.; Thomas, D. J.; Hartmann, T.; Ma, L.; Williams, A. Sci. Total Environ. 2012, 420, 334.
(29) Bach, G.; Chen, C.-S.; Pagano, R. E. Clin. Chim. Acta 1999, 280, 173.
(30) Akhmedov, D.; Braun, M.; Mataki, C.; Park, K.-S.; Pozzan, T.; Schoonjans, K.; Rorsman, P.; Wollheim, C. B.; Wiederkehr, A. The FASEB Journal 2010, 24, 4613.
(31) Lee, E. S.; Gao, Z.; Bae, Y. H. J. Controlled Release 2008, 132, 164.
(32) Schmitt-Willich, H. The British Journal of Radiology 2007, 80, 581.
(33) Zhang, X.; Lu, S.; Gao, C.; Chen, C.; Zhang, X.; Liu, M. Nanoscale 2013, 5, 6498.
(34) Li, Y.; Zhou, S. Sensors Actuators B: Chem. 2013, 177, 792.
(35) Hoare, T.; Pelton, R. The Journal of Physical Chemistry B 2007, 111, 11895.
(36) Zhao, C.; Chen, Q.; Patel, K.; Li, L.; Li, X.; Wang, Q.; Zhang, G.; Zheng, J. Soft Matter 2012, 8, 7848.
(37) Dadsetan, M.; Taylor, K. E.; Yong, C.; Bajzer, \check{Z}.; Lu, L.; Yaszemski, M. J. Acta Biomater. 2013, 9, 5438.
(38) Sorrell, C. D.; Carter, M. C. D.; Serpe, M. J. Adv. Funct. Mater. 2011, 21, 425.
(39) Islam, M. R.; Serpe, M. J. Biosens. Bioelectron. 2013, 49, 133.
(40) Islam, M. R.; Serpe, M. J. Chem. Commun. 2013, 49, 2646.
(41) Hoare, T.; Pelton, R. Macromolecules 2004, 37, 2544.
(42) Zhou, S.; Chu, B. The Journal of Physical Chemistry B 1998, 102, 1364.
(43) Dowding, P. J.; Vincent, B.; Williams, E. J. Colloid Interface Sci. 2000, 221, 268.
(44) Fernandez-Nieves, A.; Wyss, H.; Mattsson, J.; Weitz, D. A. Microgel Suspensions: Fundamentals and Applications; John Wiley & Sons, 2011.
(45) Eke, I.; Elmas, B.; Tuncel, M.; Tuncel, A. Colloids Surf Physicochem. Eng. Aspects 2006, 279, 247.
(46) Nur, H.; Pinkrah, V. T.; Mitchell, J. C.; Benée, L. S.; Snowden, M. J. Adv. Colloid Interface Sci. 2010, 158, 15.
(47) Bradley, M.; Vincent, B.; Burnett, G. Langmuir 2007, 23, 9237.
(48) Bradley, M.; Vincent, B. Langmuir 2008, 24, 2421.
(49) Smith, M. H.; Lyon, L. A. Macromolecules 2011, 44, 8154.
(50) Hoare, T.; Sivakumaran, D.; Stefanescu, C. F.; Lawlor, M. W.; Kohane, D. S. Acta Biomater. 2012, 8, 1450.
(51) Hoare, T.; Young, S.; Lawlor, M. W.; Kohane, D. S. Acta Biomater. 2012, 8, 3596.
(52) Gao, Y.; Ahiabu, A.; Serpe, M. J. ACS Appl. Mater. Interfaces 2014, 6, 13749.
(53) Thornton, P. D.; Mart, R. J.; Ulijn, R. V. Adv. Mater. 2007, 19, 1252.
(54) Thornton, P. D.; Mart, R. J.; Webb, S. J.; Ulijn, R. V. Soft Matter 2008, 4, 821.
(55) Wu, Y.; Hu, H.; Hu, J.; Liu, S. Macromol. Rapid Commun. 2012, 33, 1852.
(56) Yin, R.; Wang, K.; Du, S.; Chen, L.; Nie, J.; Zhang, W. Carbohydr. Polym. 2014, 103, 369.
(57) Hoare, T.; Pelton, R. Macromolecules 2007, 40, 670.
(58) Hoare, T.; Pelton, R. Biomacromolecules 2008, 9, 733.
(59) Matsumoto, A.; Yamamoto, K.; Yoshida, R.; Kataoka, K.; Aoyagi, T.; Miyahara, Y. Chem. Commun. 2010, 46, 2203.
(60) Matsumoto, A.; Ishii, T.; Nishida, J.; Matsumoto, H.; Kataoka, K.; Miyahara, Y. Angew. Chem. 2012, 124, 2166.
(61) Tang, Z.; Guan, Y.; Zhang, Y. Polymer Chemistry 2014, 5, 1782.
(62) Wu, Q.; Wang, L.; Yu, H.; Chen, Z. Polymer Science Series A 2012, 54, 209.
(63) Farooqi, Z. H.; Wu, W.; Zhou, S.; Siddiq, M. Macromol. Chem. Phys. 2011, 212, 1510.
(64) Farooqi, Z. H.; Khan, A.; Siddiq, M. Polym. Int. 2011, 60, 1481.
(65) Zhao, L.; Xiao, C.; Ding, J.; He, P.; Tang, Z.; Pang, X.; Zhuang, X.; Chen, X. Acta Biomater. 2013, 9, 6535.
(66) Scott, E. A.; Nichols, M. D.; Cordova, L. H.; George, B. J.; Jun, Y.-S.; Elbert, D. L. Biomaterials 2008, 29, 4481.
(67) Wang, Y.; Nie, J.; Chang, B.; Sun, Y.; Yang, W. Biomacromolecules 2013, 14, 3034.
(68) Ulasan, M.; Yavuz, E.; Bagriacik, E. U.; Cengeloglu, Y.; Yavuz, M. S. J Biomed Mater Res A 2014, DOI: 10.1002/jbm.a.35146.
(69) Binauld, S.; Stenzel, M. H. Chem. Commun. 2013, 49, 2082.
(70) Patenaude, M.; Hoare, T. ACS Macro Letters 2012, 1, 409.
(71) Smeets, N. M. B.; Bakaic, E.; Patenaude, M.; Hoare, T. Chemical Communications 2014, 50, 3306.
(72) Campbell, S. B.; Patenaude, M.; Hoare, T. Biomacromolecules 2013, 14, 644.
(73) Pawlikowska, P.; Gajkowska, B.; Orzechowski, A. Cell Tissue Res. 2007, 327, 571.
(74) Pelton, R. Adv. Colloid Interface Sci. 2000, 85, 1.
(75) Tan, S.; Zhao, D.; Yuan, D.; Wang, H.; Tu, K.; Wang, L.-Q. React. Funct. Polym. 2011, 71, 820.
(76) Lee, H.; Jeong, Y.; Park, T. G. Biomacromolecules 2007, 8, 3705.
(77) Gaulding, J. C.; Smith, M. H.; Hyatt, J. S.; Fernandez-Nieves, A.; Lyon, L. A. Macromolecules 2011, 45, 39.
(78) Bai, Y.; Zhang, Z.; Deng, M.; Chen, L.; He, C.; Zhuang, X.; Chen, X. Polym. Int. 2012, 61, 1151.
(79) Radovic-Moreno, A. F.; Lu, T. K.; Puscasu, V. A.; Yoon, C. J.; Langer, R.; Farokhzad, O. C. ACS Nano 2012, 6, 4279.
(80) Sivakumaran, D.; Maitland, D.; Hoare, T. Biomacromolecules 2011, 12, 4112.
(81) Tan, S.; Zhao, D.; Yuan, D.; Wang, H.; Tu, K.; Wang, L.-Q. React. Funct. Polym. 2011, 71, 820.
(82) Murthy, N.; Xu, M.; Schuck, S.; Kunisawa, J.; Shastri, N.; Fréchet, J. M. J. Proceedings of the National Academy of Sciences 2003, 100, 4995.
(83) Bysell, H.; Månsson, R.; Hansson, P.; Malmsten, M. Adv. Drug Del. Rev. 2011, 63, 1172.
(84) DiCesare, N.; Adhikari, D.; Heynekamp, J.; Heagy, M.; Lakowicz, J. J. Fluoresc. 2002, 12, 147.
(85) Smeets, N. M. B.; Bakaic, E.; Patenaude, M.; Hoare, T. Chemical Communications 2014, 50, 3306.
(86) Ben-Moshe, M.; Alexeev, V. L.; Asher, S. A. Anal. Chem. 2006, 78, 5149.
(87) Thaiboonrod, S.; Berkland, C.; Milani, A. H.; Ulijn, R.; Saunders, B. R. Soft Matter 2013, 9, 3920.
(88) Radovic-Moreno, A. F.; Lu, T. K.; Puscasu, V. A.; Yoon, C. J.; Langer, R.; Farokhzad, O. C. ACS Nano 2012, 6, 4279.
(89) Lutz, J.-F.; Akdemir, Ö.; Hoth, A. J Am Chem Soc 2006, 128, 13046.

1) A method for the preparation of microgel particles, the method comprising:
(a) dissolving a stimuli-responsive pre-polymer in an aqueous solvent to form an aqueous solution,
(b) applying a stimulus to the aqueous solution to form nanoaggregate pre-polymer; and
(c) adding a cross-linking agent to the aqueous solution to crosslink the nanoaggregate pre-polymer to form the microgel particles,
wherein the stimuli-responsive pre-polymer is functionalized with cross-linkable moieties.

2) The method of claim 1, wherein the stimuli-responsive pre-polymer is a thermally responsive polymer, a pH responsive polymer, an ionic strength responsive polymer or a light responsive polymer.

3) The method of claim 1, wherein the stimuli-responsive pre-polymer is functionalized with cross-linkable hydrophilic or ionic moieties.

4) The method of claim 4, wherein the stimuli-responsive pre-polymer is functionalized with a hydrazide moiety or an aldehyde moiety.

5) The method of claim 5, wherein the stimuli-responsive pre-polymer is a copolymer of
a. N-isopropylacrylamide, poly(oligoethylene glycol methacrylate) or polyvinylcaprolactam; and
b. a second monomer which can be functionalized with a hydrazide moiety or an aldehyde moiety.

6) The method of claim 5, wherein the second monomer is acrylic acid, methacrylic acid, itaconic acid, fumaric acid, maleic acid, vinylacetic acid, or a derivative thereof or N-(2,2-dimethoxyethyl)methacrylamide (DMEMAm) or a derivative thereof.

7) The method of claim 5, wherein the copolymer further comprises monomers which are BAHC or DMAEMA.

8) The method of claim 1, wherein the microgel particles are degradable in vivo to reform the stimulus-responsive pre-polymer.

9) The method of claim 8, wherein the stimuli-responsive pre-polymer has a molecular weight lower than about 60 kDa.

10) The method of claim 1, wherein the stimulus is a change in temperature, pH, ionic strength, or light conditions.

11) The method of claim 10, wherein the stimulus is a change in temperature.

12) The method of claim 11, wherein the stimulus is an increase in temperature above the lower critical solution temperature (LCST) of the stimulus-responsive polymer.

13) The method of claim 1, wherein the cross-linking polymer is functionalized with moieties which are complementary to the cross-linkable moieties of the stimulus-responsive polymer.

14) The method of claim 13, wherein the covalent cross-links are formed via reaction of hydrazide and aldehyde groups to form reversible hydrazone bonds.

15) The method of claim 13, wherein the cross-linking polymer is a copolymer, comprising:
a) a first monomer which is (N-isopropylacrylamide) (NIPAM), (oligo(ethylene glycol)methacrylate) or vinylcaprolactam; and
b) a second monomer which can be functionalized with cross-linkable moieties which are complementary to the cross-linkable moieties of the stimuli-responsive pre-polymer.

16) A composition comprising microgel particles, wherein the microgel particles comprise:
a. discrete nanoaggregate particles comprised of stimuli-responsive pre-polymers, wherein the nanoaggregate particles are cross-linked with a cross-linking polymer;
wherein the microgel particles are biodegradable in vivo to reform the stimuli-responsive pre-polymers;
wherein the stimuli-responsive pre-polymers have a molecular weight lower than the renal clearance cut-off, and
wherein the composition is substantially free of organic solvents.

17) The composition according to claim 16, wherein the stimuli-responsive pre-polymers have a molecular weight lower than 60 kDa.

18) The composition according to claim 16, wherein the stimuli-responsive pre-polymer is a copolymer of
a. N-isopropylacrylamide, poly(oligoethylene glycol methacrylate) or polyvinylcaprolactam; and
b. a second monomer which can be functionalized with a hydrazide moiety or an aldehyde moiety.
The composition of claim 18, wherein the second monomer is acrylic acid, methacrylic acid, itaconic acid, fumaric acid, maleic acid, vinylacetic acid, or a derivative thereof or N-(2,2-dimethoxyethyl)methacrylamide (DMEMAm) or a derivative thereof.
